SEQUENCE LISTING (1 sequence)

SEQ ID NO: 1 - DNA, 9313 bp, synthetic construct
  source 1..9313: mol_type = other DNA
ttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccagaccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggcgccggctcgagtggctccggtgcccgtcagtgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattctgcagtcgacgccgccaccatgagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaatcccgctagcggatcgggagagggcagaggaagtctgctaacatgcggtgacgtcgaggagaatcctggcccaccggtcgccaccgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacccacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgaacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtacgtatagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagc

(19) United States
(12) Patent Application Publication
(10) Pub. No.: US 2026/0317756 A1
(43) Pub. Date: Oct. 15, 2026

(54) ENHANCED MAMMALIAN CRISPR EDITING WITH SEPARATED RETRON DONOR AND NICKASES

(71) Applicants: The Board Of Trustees Of The Leland Stanford Junior University, Stanford, CA (US); European Molecular Biology Laboratory, Heidelberg (DE)

(72) Inventors: Dominik Lindenhofer, Heidelberg (DE); Kevin R. Roy, Stanford, CA (US); Lars M. Steinmetz, Stanford, CA (US)

(21) Appl. No.: 19/104,501

(22) PCT Filed: Aug. 25, 2023
(86) PCT No.: PCT/US2023/072893

§ 371 Date: Feb. 18, 2025

Related U.S. Application Data
(60) Provisional application No. 63/400,952, filed on Aug. 25, 2022.

(51) Int. Cl. C12N 15/11; C12N 9/22; C12N 15/10; C12N 15/81; C12N 15/90

(52) U.S. Cl. CPC C12N 15/11; C12N 9/226; C12N 15/102; C12N 15/81; C12N 15/907; C12N 2310/20

(57) ABSTRACT

Described herein is a CRISPR guide and retron donor expressed separately to mediate genome editing by homology-directed repair. The split system works more efficiently than previous approaches employing retron-gRNA fusions, demonstrating that recruitment of the retron to the target site by Cas9 is not essential for retron-based editing and that retron and guide functionality can be enhanced when expressed via distinct promoters and RNA processing elements. More efficient editing was achieved with a mutant Cas9 nickase than with the fully active Cas9 nuclease in mammalian cells, avoiding the toxicity and error-prone repair pathways triggered by double-strand breaks which are of major concern for therapeutic applications.

### CROSS-REFERENCE TO RELATED APPLICATIONS

This application claims priority to U.S. Provisional Application No. 63/400,952, filed Aug. 25, 2022, the disclosure of which is herein incorporated by reference in its entirety for all purposes.

### STATEMENT AS TO RIGHTS TO INVENTIONS MADE UNDER FEDERALLY SPONSORED RESEARCH AND DEVELOPMENT

This invention was made with Government support under Contract No. R01GM121932 awarded by the National Institutes of Health. The Government has certain rights in the invention.

### BACKGROUND

In most organisms and cell types, single-stranded DNA (ssDNA) serves as a more efficient donor template for homology-directed repair (HDR) than double-stranded DNA (dsDNA). This is likely due to the better availability of ssDNA for mediating HDR, as it can directly base-pair with the target edit site without requiring helicase activity needed to unwind dsDNA. While ssDNA can be delivered to cells as synthetic oligonucleotides, this approach is not optimal in many settings because (i) the ssDNA must pass through the cell membrane and cell nucleus where editing takes place, (ii) it is diluted by cell growth and turnover due to nucleases, and (iii) it is not amenable to multiplexed editing approaches, where each cell needs to receive different donor paired with a CRISPR guide RNA. Recent work has addressed all three challenges by harnessing an in vivo ssDNA production system evolved by bacteria called retrons for editing in bacterial and mammalian cells (Farzadfard et al., 2014; Sharon et al., 2018; Lopez et al., 2022).

Bacterial retrons are a two-component system, a retron possessing reverse transcriptase (RT) activity, and a non-coding transcript termed the msr-msd RNA which serves as a template for the RT (Simon et al., 2019). The retron RT recognizes a specific secondary structure on the msr-msd transcript and reverse transcribes the msd part of the transcript, resulting in an RNA-DNA hybrid product termed multicopy single-stranded DNA (msDNA). Critically, the msd part can be programmed to contain arbitrary sequence by inserting the desired sequence into a loop region of the msd component of the retron msr-msd transcript (Farzadfard et al., 2014). This allows for production of single-stranded donor DNA for HDR intracellularly, holding potential to improve editing efficiency. The fusion approach was first employed in yeast, where the msr-msd-donor was fused to the 5′ end of the gRNAs in an effort to simplify the co-delivery of both donor and guide and to further have the Cas9::guide complex bring the donor to the edit site for enhanced HDR efficiency (FIG. 1A; Sharon et al., 2018). This approach has recently been employed in mammalian cells with several variations at the level of testing retrons from diverse bacteria, utilizing different promoters, alteration of retron structure, and retron-gRNA fusion orientation (Kong et al., 2021; Lopez et al., 2021; Zhao et al., 2022). To date, the highest efficiencies have been achieved with the Ec73 retron RT system (also known as Retron-Eco3) and with the msr-msd-donor fused to the 3′ end of the gRNAs and driven from a RNA Polymerase II promoter (FIG. 2A; Kong et al., 2021). These constructs also use a hammerhead ribozyme (HHR) at the 5′ end and a hepatitis delta virus (HDV) ribozyme at the 3′ end to precisely release both gRNA and msr-msd-donor of the primary RNA polymerase II transcript and prevent nuclear export.

The present disclosure provides a more efficient retron editing system that does not require the creation of double-strand breaks.

### BRIEF SUMMARY

Provided are compositions and methods for gene editing in mammalian cells. In one aspect the disclosure provides a nucleic acid comprising: (i) a first promoter operably linked to a retron nucleic acid sequence comprising: an msr sequence; an msd sequence; a donor sequence within the msd sequence; and a first inverted repeat sequence and a second inverted repeat sequence; and (ii) a second promoter operably linked to a nucleic acid sequence encoding a guide RNA region.

In some embodiments, the first promoter comprises an RNA polymerase II (Pol II) promoter, and the second promoter comprises an RNA polymerase III (Pol III) promoter.

In some embodiments, the nucleic acid further comprises a stabilizing 5′ ribozyme sequence located 3′ of the first promoter and 5′ of the first inverted repeat sequence; a stabilizing 3′ ribozyme sequence located 3′ of the second inverted repeat sequence; and/or a long non-coding RNA (lncRNA) transcript located 3′ of the second inverted repeat sequence.

In some embodiments, the stabilizing 5′ or 3′ ribozyme sequence is selected from the group consisting of a hammerhead ribozyme (HHR), a hepatitis delta virus (HDV) ribozyme, and a RiboJ ribozyme sequence.

In some embodiments, the stabilizing 3′ sequence comprises the 3′ triple-helix and tRNA-like processing components of the lncRNA transcript Metastasis Associated Lung Adenocarcinoma Transcript 1 (MALATI).

In some embodiments, the nucleic acid further comprises a transcription terminator sequence located 3′ of the second inverted repeat sequence and 5′ of the second promoter.

In some embodiments, the donor sequence comprises a template for homology-directed repair (HDR).

In another aspect, the disclosure provides a nucleic acid comprising a transcription unit comprising a promoter operably linked to a nucleic acid sequence encoding a retron RNA and a nucleic acid sequence encoding a guide RNA region, the retron nucleic acid sequence comprising: an msr sequence; an msd sequence; a donor sequence within the msd sequence; and a first inverted repeat sequence and a second inverted repeat sequence.

In some embodiments, expression of the nucleic acid sequence results in a transcript comprising the retron RNA and guide RNA region, wherein the retron RNA and guide RNA region are separated after transcription by an RNA processing enzyme. In some embodiments, the RNA processing enzyme is a ribozyme or an endoribonuclease. In some embodiments, the promoter comprises an RNA polymerase II (Pol II) promoter or an RNA polymerase III (Pol III) promoter. In some embodiments, the nucleic acid sequence encoding the retron RNA is located 5′ of the nucleic acid sequence encoding the guide RNA. In some embodiments, the nucleic acid sequence encoding the retron RNA is located 3′ of the nucleic acid sequence encoding the guide RNA.

In another aspect, the disclosure provides an expression plasmid comprising a nucleic acid described herein.

In another aspect, the disclosure provides a system for introducing a genetic modification at a target DNA locus, the system comprising: i) a first expression plasmid comprising a nucleic acid described herein, ii) a second expression plasmid comprising a nucleic acid sequence encoding a CRISPR-associated endonuclease; and iii) a third expression plasmid comprising a nucleic acid sequence encoding a reverse transcriptase.

In some embodiments, the second and third expression plasmids comprise a promoter operably linked to the nucleic acid sequences encoding the CRISPR-associated endonuclease and the reverse transcriptase, respectively. In some embodiments, the promoter is a Pol II promoter.

In some embodiments, the CRISPR-associated endonuclease generates a single-stranded nick in one strand of the target DNA locus instead of a double-strand break (DSB). In some embodiments, the CRISPR-associated endonuclease comprises a nickase selected from the group consisting of nCas9-D10A and nCas9-H840A.

In some embodiments, the reverse transcriptase is selected from the group consisting of RT-Ec73, RT-St85, RT-Ec83, RT-Ec78, RT-Eco9, RT-Eco10, and RT-Eco11. In some embodiments, the reverse transcriptase is RT-Ec73.

In another aspect, the disclosure provides a method for editing DNA at a target locus in a cell, the method comprising introducing a system of the disclosure into the cell.

In some embodiments, the editing efficiency at the target locus is increased compared to a system comprising an expression plasmid wherein transcription products of the retron and the gRNA coding region are physically coupled.

In some embodiments, the cell is selected from the group consisting of a yeast cell, plant cell, mammalian cell, mammalian cell line, human cell, and human cell line.

In another aspect, the disclosure provides a method of treating a genetic disease in a subject in need thereof, the method comprising administering to the subject an effective amount of a) a nucleic acid of the disclosure, a plasmid comprising a nucleic acid of the disclosure, or a system of the disclosure, or a combination thereof; b) a reverse transcriptase or a nucleic acid encoding the same, and c) a sequence-specific endonuclease or a nucleic acid encoding the same.

In some embodiments, the sequence-specific endonuclease does not catalyze a double strand break in the DNA of a host cell in the subject.

In some embodiments, the sequence-specific endonuclease generates a single-stranded nick in one strand of a target DNA locus in a host cell of the subject. In some embodiments, the sequence-specific endonuclease comprises a nickase selected from the group consisting of nCas9-D10A and nCas9-H840A. In some embodiments, the nickase is nCas9-H840A.

In another aspect, described herein is a pharmaceutical composition comprising: (a) a nucleic acid of the disclosure, a plasmid comprising a nucleic acid of the disclosure, or a system of the disclosure, or a combination thereof, and (b) a pharmaceutically acceptable carrier.

In another aspect, described herein is a method for preventing or treating a genetic disease in a subject, the method comprising administering to the subject an effective amount of a pharmaceutical composition of the disclosure to correct a mutation in a target gene associated with the genetic disease.

In some embodiments, the genetic disease is selected from the group consisting of X-linked severe combined immune deficiency, sickle cell anemia, thalassemia, hemophilia, neoplasia, cancer, age-related macular degeneration, schizophrenia, trinucleotide repeat disorders, fragile X syndrome, prion-related disorders, amyotrophic lateral sclerosis, drug addiction, autism, Alzheimer's disease, Parkinson's disease, cystic fibrosis, blood and coagulation diseases and disorders, inflammation, immune-related diseases and disorders, metabolic diseases and disorders, liver diseases and disorders, kidney diseases and disorders, muscular/skeletal diseases and disorders, neurological and neuronal diseases and disorders, cardiovascular diseases and disorders, pulmonary diseases and disorders, ocular diseases and disorders, and a combination thereof.

In another aspect, the disclosure provides a kit for modifying one or more target nucleic acids of interest at one or more target loci within a genome of a host cell, the kit comprising a nucleic acid of the disclosure, a plasmid comprising a nucleic acid of the disclosure, or a system of the disclosure, or a combination thereof.

In some embodiments, the kit comprises a reverse transcriptase or a nucleic acid encoding the same, and a sequence-specific endonuclease or a nucleic acid encoding the same. In some embodiments, the reverse transcriptase included in the kit is selected from the group consisting of RT-Ec73, RT-St85, RT-Ec83, RT-Ec78, RT-Eco9, RT-Eco10, and RT-Eco11. In some embodiments, the reverse transcriptase is RT-Ec73. In some embodiments, the sequence-specific endonuclease comprises a nickase selected from the group consisting of nCas9-D10A and nCas9-H840A. In some embodiments, the nickase is nCas9-H840A.

In some embodiments, the kit comprises a host cell. In some embodiments, the host cell is selected from the group consisting of a yeast cell, a plant cell, mammalian cell, mammalian cell line, human cell, and human cell line. In some embodiments, the kit comprises one or more reagents for introducing the nucleic acids, plasmids, reverse transcriptase or sequence-specific endonuclease into the host cell.

In another aspect, the disclosure provides a method for producing a retron RNA and a guide RNA, the method comprising: i) contacting the nucleic acid of any one of claims 8 to 11 with an RNA polymerase to produce a single transcript comprising the retron RNA sequences and guide RNA sequences, and ii) contacting the single transcript with an RNA processing enzyme that cleaves the transcript between the retron RNA and guide RNA sequences, thereby producing the retron RNA and the guide RNA.

In some embodiments, the RNA processing enzyme is a ribozyme or an endoribonuclease.

In some embodiments, the nucleic acid sequence encoding the retron RNA is located 5′ of the nucleic acid sequence encoding the guide RNA. In some embodiments, the nucleic acid sequence encoding the retron RNA is located 3′ of the nucleic acid sequence encoding the guide RNA.

### BRIEF DESCRIPTION OF THE DRAWINGS

FIGS. 1A-ID: (A) Outline of the retron donor-guide fusion (rgRNA) setup for editing in the budding yeast Saccharomyces cerevisiae. The transcript is expressed from the S. cerevisiae GAL7 promoter, which will undergo cleavage by either a 5′ HHR or a 5′ RiboJ ribozyme and a 3′ HDV ribozyme. This releases the rgRNA from the 5′ cap and 3′ poly (A) tail elements, respectively, which would otherwise promote mRNA export from the nucleus in eukaryotic cells. (B) Outline of the split gRNA::retron-donor system. gRNA expression is driven by the S. cerevisiae SNR52 promoter, and Ec86 msr-msd-donor expression is driven by the GAL7 promoter. The 5′ HDV ribozyme confers greater retron cDNA production (U.S. provisional No. 63/214,197). (C) Fully processed transcripts generated from the 5′ HHR rgRNA fusion (left), 5′ riboJ rgRNA fusion (middle), and split 5′ HDV retron and SNR52 guide setups (right) are shown. (D) On-target editing outcomes at the ADE2 locus using a previously published 5′ HHR rgRNA fusion cassette as a reference (Sharon et al., Cell, 2018). Both SpCas9 and the Ec86 reverse transcriptase (RT) are expressed from the bi-directional S. cerevisiae GALI/GALIO promoter. Editing efficiency was quantified by amplicon sequencing of the ADE2 target locus. Non-homologous end joining-mediated insertions or deletions (NHEJ indels) were identified as small indels at the Cas9 cleavage site.

FIGS. 2A-2D: (A) Outline of the gRNA-msr-msd-donor (rgRNA) fusion. The rgRNA transcript from a Pol-II promoter is cleaved by a 5′ HHR and a 3′ HDV ribozyme to release rgRNA. (B) Outline of the split gRNA:msr-msd-donor system. gRNA expression is driven by a U6 promoter, msr-msd-donor expression is driven by a Pol-II promoter. Various ribozyme structures are added on the S′ and 3′ end of the msr-msd-donor transcript to make the msr-msd-donor transcript more stable within cells. (C) Scheme of the HDR reporter, leveraging retron-generated intracellular ssDNA with a Cas9-induced cleavage at the target site. EGFP reconstitution using transgenic HEK293 cells expressing TagBFP-P2A-nEGFP is used to determine HDR efficiencies by flow cytometry. (D) Scheme of the mutation made to report for EGFP fluorescence. The mutation is a C->T substitution, with an additional silent mutation of the PAM motif to prevent gRNAs from recutting the site after editing and to prevent degradation of the plasmid harboring the msr-msd-donor.

FIGS. 3A-3F: (A) Outline of the lipofection experiment for testing the fusion versus split rgRNA systems and WT Cas9 versus Cas9 nickases. Lipofected cells are indicated with a black dot; EGFP positive cells are indicated white. (B) Outline of the msDNA-donor generated and the homology arms used for editing. (C) Outline of the constructs used for testing the rgRNA fusion previously published using the Ec73 retron RT (Kong et al., 2021). (D) Outline of the constructs used to test the split gRNA::msr-msd-donor system. Data for the rgRNA fusion (E) and the split gRNA::msr-msd-donor system (F). The density of the grey color indicates percentage of normalized GFP positive cells. Summary bar graph data of the aggregated data from each heatmap above is shown at the bottom. Data bars are mean±SEM.

FIGS. 4A-4D: (A) Outline of the lipofection experiment for testing the alternative retrons for promoting HDR. Lipofected cells are indicated with a black dot; EGFP positive cells are indicated white. (B) Outline of the msDNA-donor generated and the homology arms used for editing. (C) Outline of the constructs used for testing the alternative retrons. The msr-msd sequence was adjusted for the respective retron in plasmid 1. Retrons tested were Eco4, Eco7, Eco9, Eco10, Eco11 and Sen2. (D) Data for the testing of alternative retrons. Different 5′ and 3′ elements of the msr-msd transcript are outlined on the left. Black rectangles indicate absence of these conditions. The density of the grey color indicates percentage of normalized GFP positive cells. Summary bar graph data of the aggregated data from the heatmap is shown at the bottom highlighting the effect of different Cas9 nuclease versions on editing performance. Data bars are mean ±SEM.

### DETAILED DESCRIPTION

Provided herein are compositions and methods for gene editing in mammalian cells. The disclosure describes the use of a CRISPR guide and retron donor expressed separately to mediate genome editing by homology-directed repair. The inventors show that this “split” system works more efficiently than previous approaches employing retron donor-gRNA fusions, demonstrating that retron and guide functionality can be enhanced when expressed via distinct promoters and/or RNA processing elements. The disclosure demonstrates that in both yeast and mammalian cells, the split system is more efficient than the previously published fusion approaches, contradicting the model that the retron donor must be recruited by Cas9 for efficient HDR.

The inventors also demonstrate more efficient editing with the genetically modified Cas9 nickases than with the fully active Cas9 nuclease in mammalian cells. Editing with nickases has the additional advantage of avoiding the toxicity and error-prone repair pathways triggered by double-strand breaks, which are of major concern for therapeutic applications. The inventors have shown that the split approach provides superior editing with Cas9 nickases, such as nCas9-D10A and nCas9-H840A, bypassing the levels observed with fully active Cas9 and retron-gRNA (rgRNA) fusions. By contrast, the rgRNA fusions lack detectable editing with nickases. The split retron donor-guide and nickase approach allows for precise editing while reducing formation of indels and other unwanted on-target and off-target effects.

The disclosure provides advantages over previous gene editing technologies, enabling i) a system amenable for efficient single-plex and multiplexed genome editing, ii) enhanced editing capabilities in mammalian cells, and iii) a physically separated retron and gRNA make the system more versatile to use as each RNA can be expressed from optimal promoters, referred to herein as a “split” system. The split retron donor-guide approach with Cas9 nickase has a similar editing efficiency compared to a published rgRNA fusion approach which used double-strand break Cas9 (Kong et al., 2021). This is a major advantage as nickases induce far fewer unintended edits at both the on- and off-target sites. The previously developed fusion rgRNA approaches show little editing with Cas9 nickases, such as nCas9-D10A and nCas9-H840A, demonstrating that the retron donor technology is important for efficient HDR using nickases.

The practice of the present disclosure employs, unless otherwise indicated, conventional techniques of immunology, biochemistry, chemistry, molecular biology, microbiology, cell biology, genomics and recombinant DNA, which are within the skill of the art. See Sambrook, Fritsch and Maniatis, Molecular Cloning: A Laboratory Manual, 4th edition (2012), Current Protocols in Molecular Biology (F. M. Ausubel, et al. eds., (2012)), the series Methods in Enzymology (Academic Press, Inc.), and PCR 2: A Practical Approach (M. J. MacPherson, B. D. Hames and G. R. Taylor eds. (1995)).

For nucleic acids, sizes are given in either kilobases (kb), base pairs (bp), or nucleotides (nt). Sizes of single-stranded DNA and/or RNA can be given in nucleotides. These are estimates derived from agarose or acrylamide gel electrophoresis, from sequenced nucleic acids, or from published DNA sequences. For proteins, sizes are given in kilodaltons (kDa) or amino acid residue numbers. Protein sizes are estimated from gel electrophoresis, from sequenced proteins, from derived amino acid sequences, or from published protein sequences.

Oligonucleotides that are not commercially available can be chemically synthesized, e.g., according to the solid phase phosphoramidite triester method first described by Beaucage and Caruthers, Tetrahedron Lett. 22:1859-1862 (1981), using an automated synthesizer, as described in Van Devanter et. al., Nucleic Acids Res. 12:6159-6168 (1984). Purification of oligonucleotides is performed using any art-recognized strategy, e.g., native acrylamide gel electrophoresis or anion-exchange high performance liquid chromatography (HPLC) as described in Pearson and Reanier, J. Chrom. 255:137-149 (1983).

### I. Definitions

It is to be understood that this disclosure is not strictly limited to particular embodiments described, as such may of course vary. It is also to be understood that the terminology used herein is for the purpose of describing particular embodiments only, and is not intended to be limiting, since the scope of the present disclosure will be limited only by the claims.

As used herein and in the appended claims, the singular forms “a,” “an,” and “the” include plural referents unless the context clearly dictates otherwise. It should further be understood that as used herein, the term “a” entity or “an” entity refers to one or more of that entity. For example, a nucleic acid molecule refers to one or more nucleic acid molecules. As such, the terms “a”, “an”, “one or more” and “at least one” can be used interchangeably. Similarly, the terms “comprising”, “including” and “having” can be used interchangeably.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

It is appreciated that certain features of the disclosure, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments are specifically embraced by the present invention and are disclosed herein just as if each and every combination were individually and explicitly disclosed. In addition, all sub-combinations are also specifically embraced by the present invention and are disclosed herein just as if each and every such sub-combination were individually and explicitly disclosed herein.

It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As used herein, the term “about” means a range of values including the specified value, which a person of ordinary skill in the art would consider reasonably similar to the specified value. In some embodiments, about means within a standard deviation using measurements generally acceptable in the art. In some embodiments, about means a range extending to +/−10% of the specified value (e.g., +/−1%, 2%, 3%, 4%, 5%, 6%, 7%, 8%, 9%, or 10% of the specified value). In some embodiments, about means the specified value.

The term “genome editing” refers to a type of genetic engineering in which DNA is inserted, replaced, or removed from a target DNA (e.g., the genome of a cell) using one or more nucleases and/or nickases. The nucleases create specific double-strand breaks (DSBs) at desired locations in the genome and harness the cell's endogenous mechanisms to repair the induced break by homology-directed repair (HDR) (e.g., homologous recombination) or by nonhomologous end joining (NHEJ). The nickases create specific single-strand breaks at desired locations in the genome. In one non-limiting example, two nickases can be used to create two single-strand breaks on opposite strands of a target DNA, thereby generating a blunt or a sticky end. Any suitable DNA nucleases and/or nickases can be introduced into a cell to induce genome editing of a target DNA sequence.

As used herein, the term “retron” is used in accordance with its plain ordinary meaning and refers to a DNA sequence found in the genome of many bacteria species that codes for reverse transcriptase and a unique single-stranded DNA/RNA hybrid called multicopy single-stranded DNA (msDNA). The retron msr-msd RNA is the non-coding RNA produced by retron elements and is the immediate precursor to the synthesis of msDNA. The retron msr RNA folds into a characteristic secondary structure that contains a conserved guanosine residue at the end of a stem loop. Synthesis of DNA by the retron-encoded reverse transcriptase (RT) results in a DNA/RNA chimera which is composed of small single-stranded DNA linked to small single-stranded RNA. The RNA strand is joined to the 5′ end of the DNA chain via a 2′-5′ phosphodiester linkage that occurs from the 2′ position of the conserved internal guanosine residue. The retron operon carries a promoter sequence P that controls the synthesis of an RNA transcript carrying three loci: msr, msd, and ret. The ret gene product, a reverse transcriptase, processes the msd/msr portion of the RNA transcript into msDNA. Retron elements are about 2 kb long. They contain a single operon controlling the synthesis of an RNA transcript carrying three loci, msr, msd, and ret, that are involved in msDNA synthesis. The DNA portion of msDNA is encoded by the msd region, the RNA portion is encoded by the msr region, while the product of the ret open-reading frame is a reverse transcriptase similar to the RTs produced by retroviruses and other types of retroelements. Like other reverse transcriptases, the retron RT contains seven regions of conserved amino acids, including a highly conserved tyr-ala-asp-asp (YADD) sequence associated with the catalytic core. The ret gene product is responsible for processing the msd/msr portion of the RNA transcript into msDNA. When utilized in eukaryotic cells, the coding ret gene and the non-coding RNA msr-msd are optimally expressed from separate promoters.

As used herein, the term “reverse transcriptase” refers to its plain and ordinary meaning as an enzyme used to generate complementary DNA (cDNA) from an RNA template, a process termed reverse transcription.

As used herein, the terms “complementary” or “complementarity” refers to polynucleotides that are able to form base pairs with one another. Base pairs are typically formed by hydrogen bonds between nucleotide units in an anti-parallel orientation between polynucleotide strands. Complementary polynucleotide strands can base pair in a Watson-Crick manner (e.g., A to T, A to U, C to G), or in any other manner that allows for the formation of duplexes. As persons skilled in the art are aware, when using RNA as opposed to DNA, uracil (U) rather than thymine (T) is the base that is considered to be complementary to adenosine. However, when a uracil is denoted in the context of the present disclosure, the ability to substitute a thymine is implied, unless otherwise stated. “Complementarity” may exist between two RNA strands, two DNA strands, or between a RNA strand and a DNA strand. It is generally understood that two or more polynucleotides may be “complementary” and able to form a duplex despite having less than perfect or less than 100% complementarity. Two sequences are “perfectly complementary” or “100% complementary” if at least a contiguous portion of each polynucleotide sequence, comprising a region of complementarity, perfectly base pairs with the other polynucleotide without any mismatches or interruptions within such region. Two or more sequences are considered “perfectly complementary” or “100% complementary” even if either or both polynucleotides contain additional non-complementary sequences as long as the contiguous region of complementarity within each polynucleotide is able to perfectly hybridize with the other. “Less than perfect” complementarity refers to situations where less than all of the contiguous nucleotides within such region of complementarity are able to base pair with each other. Determining the percentage of complementarity between two polynucleotide sequences is a matter of ordinary skill in the art. For purposes of Cas9 targeting, a gRNA may comprise a sequence “complementary” to a target sequence (e.g., major or minor allele), capable of sufficient base-pairing to form a duplex (i.e., the gRNA hybridizes with the target sequence). Additionally, the gRNA may comprise a sequence complementary to a sequence adjacent to a PAM sequence, wherein the gRNA also hybridizes with the sequence adjacent to a PAM sequence in a target DNA.

The terms “hybridize” and “hybridization” refer to the formation of complexes between nucleotide sequences which are sufficiently complementary to form duplexes via Watson-Crick base pairing.

The term “DNA nuclease” refers to an enzyme capable of cleaving the phosphodiester bonds between the nucleotide subunits of DNA and may be an endonuclease or an exonuclease. According to the present invention, the DNA nuclease may be an engineered (e.g., programmable or targetable) DNA nuclease which can be used to induce genome editing of a target DNA sequence. Any suitable DNA nuclease can be used including, but not limited to, CRISPR-associated protein (Cas) nucleases, other endo- or exo-nucleases, variants thereof, fragments thereof, and combinations thereof.

The term “nickase” refers to an enzyme that cuts one strand of a double-stranded DNA molecule. The term includes Cas9 nickase that can be paired with a guide RNA for site-specific cleavage of a target DNA strand. A Cas9 nickase (nCas9) has only one active functional domain and can cut only one strand of the target nucleic acid, thereby creating a single strand break or nick. In some embodiments, a Cas9 nickase is a mutant Cas9 nuclease having one or more amino acid mutations. In some embodiments, the Cas9 nickase is a nCas9 D10A mutant. In other embodiments, the Cas9 nickase is a nCas9 H840A mutant. Other examples of Cas9 nickases include, without limitation, nCas9 N854A and nCas9 N863A mutants. A double-strand break can be introduced using a Cas9 nickase if at least two gRNAs that target opposite DNA strands are used. The nickases (e.g., Cas9 nickases) can be codon-optimized for the target cell or target organism.

The term “double-strand break” or “DSB” or “double-strand cut” refers to the severing or cleavage of both strands of the DNA double helix. The DSB may result in cleavage of both stands at the same position leading to “blunt ends” or staggered cleavage resulting in a region of single-stranded DNA at the end of each DNA fragment, or “sticky ends”. A DSB may arise from the action of one or more DNA nucleases.

The term “nonhomologous end joining” or “NHEJ” refers to a pathway that repairs double-strand DNA breaks in which the break ends are directly ligated without the need for a homologous template.

The term “homology-directed repair” or “HDR” refers to a mechanism in cells to accurately and precisely repair double-strand DNA breaks using a homologous template to guide repair. The most common form of HDR is homologous recombination (HR), a type of genetic recombination in which nucleotide sequences are exchanged between two similar or identical molecules of DNA.

The term “nucleic acid,” “nucleotide,” or “polynucleotide” refers to deoxyribonucleic acids (DNA), ribonucleic acids (RNA) and polymers thereof in either single-, double- or multi-stranded form. The term includes, but is not limited to, single-, double- or multi-stranded DNA or RNA, genomic DNA, cDNA, DNA-RNA hybrids, or a polymer comprising purine and/or pyrimidine bases or other natural, chemically modified, biochemically modified, non-natural, synthetic or derivatized nucleotide bases. In some embodiments, a nucleic acid can comprise a mixture of DNA, RNA and analogs thereof. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The term “single nucleotide polymorphism” or “SNP” refers to a change of a single nucleotide within a polynucleotide, including within an allele. This can include the replacement of one nucleotide by another, as well as the deletion or insertion of a single nucleotide. Most typically, SNPs are biallelic markers although tri- and tetra-allelic markers can also exist. By way of non-limiting example, a nucleic acid molecule comprising SNP AIC may include a C or A at the polymorphic position.

The term “gene” means the segment of DNA involved in producing a ribonucleic acid polymer, which in the case of protein coding genes can then be translated into a polypeptide chain. The DNA segment may include regions preceding and following the coding region (leader and trailer) involved in the transcription/translation of the gene product and the regulation of the transcription/translation, as well as intervening sequences (introns) between individual coding segments (exons).

The terms “plasmid” and “expression plasmid” refer to a recombinant circular extrachromosomal DNA molecule comprising nucleic acid sequences for replication in a host cell. A plasmid can contain regulatory elements such as promoters, enhancers, transcription terminators and polyA sequences for regulating transcription and/or translation of a heterologous sequence that is inserted or “cloned” into the plasmid. A plasmid can also include a selectable marker, such as an antibiotic resistance gene, to maintain the plasmid during culture, for example, in bacterial cells.

The term “cassette” refers to a combination of genetic sequence elements that may be introduced as a single element and may function together to achieve a desired result. A cassette typically comprises polynucleotides in combinations that are not found in nature.

The term “operably linked” refers to two or more genetic elements, such as a polynucleotide sequence and a promoter, placed in relative positions that permit the proper biological functioning of the elements, such as the promoter directing transcription of the polynucleotide sequence.

The term “inducible promoter” refers to a promoter that responds to environmental factors and/or external stimuli that can be artificially controlled in order to modify the expression of, or the level of expression of, a polynucleotide sequence or refers to a combination of elements, for example an exogenous promoter and an additional element such as a trans-activator operably linked to a separate promoter. An inducible promoter may respond to abiotic factors such as oxygen levels or to chemical or biological molecules. In some embodiments, the chemical or biological molecules may be molecules not naturally present in humans.

The terms “vector” and “expression vector” refer to a nucleic acid construct, generated recombinantly or synthetically, with a series of specified nucleic acid elements that permit transcription of a particular polynucleotide sequence in a host cell. An expression vector may be part of a plasmid, viral genome, or nucleic acid fragment. Typically, an expression vector includes a polynucleotide to be transcribed, operably linked to a promoter. The term “promoter” is used herein to refer to an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. Other elements that may be present in an expression vector include those that enhance transcription (e.g., enhancers) and terminate transcription (e.g., terminators).

“Recombinant” refers to a genetically modified polynucleotide, polypeptide, cell, tissue, or organism. For example, a recombinant polynucleotide (or a copy or complement of a recombinant polynucleotide) is one that has been manipulated using well known methods. A recombinant expression cassette comprising a promoter operably linked to a second polynucleotide (e.g., a retron guide or donor sequence, or a protein coding sequence) can include a promoter that is beterologous to the second polynucleotide as the result of human manipulation (e.g., by methods described in Sambrook et al., Molecular Cloning-A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)). A recombinant expression cassette (or expression vector) typically comprises polynucleotides in combinations that are not found in nature. For instance, human manipulated restriction sites or plasmid vector sequences can flank or separate the promoter from other sequences. A recombinant protein is one that is expressed from a recombinant polynucleotide, and recombinant cells, tissues, and organisms are those that comprise recombinant sequences (polynucleotide and/or polypeptide).

As used herein, the term “heterologous” refers to biological material that is introduced, inserted, or incorporated into a recipient (e.g., host) organism that originates from another organism. Typically, the heterologous material that is introduced into the recipient organism (e.g., a host cell) is not normally found in that organism. Heterologous material can include, but is not limited to, nucleic acids, amino acids, peptides, proteins, and structural elements such as genes, promoters, and cassettes. A host cell can be, but is not limited to, a bacterium, a yeast cell, a mammalian cell, or a plant cell. The introduction of heterologous material into a host cell or organism can result, in some instances, in the expression of additional heterologous material in or by the host cell or organism. As a non-limiting example, the transformation of a yeast host cell with an expression vector that contains DNA sequences encoding a bacterial protein may result in the expression of the bacterial protein by the yeast cell. The incorporation of heterologous material may be permanent or transient. Also, the expression of heterologous material may be permanent or transient.

The terms “reporter” and “selectable marker” can be used interchangeably and refer to a gene product that permits a cell expressing that gene product to be identified and/or isolated from a mixed population of cells. Such isolation might be achieved through the selective killing of cells not expressing the selectable marker, which may be, as a non-limiting example, an antibiotic resistance gene. Alternatively, the selectable marker may permit identification and/or subsequent isolation of cells expressing the marker as a result of the expression of a fluorescent protein such as GFP or the expression of a cell surface marker which permits isolation of cells by fluorescence-activated cell sorting (FACS), magnetic-activated cell sorting (MACS), or analogous methods. Suitable cell surface markers include CD8, CD19, and truncated CD19. Preferably, cell surface markers used for isolating desired cells are non-signaling molecules, such as subunit or truncated forms of CD8, CD19, or CD20. Suitable markers and techniques are known in the art.

The terms “culture,” “culturing,” “grow,” “growing,” “maintain,” “maintaining,” “expand,” “expanding,” etc., when referring to cell culture itself or the process of culturing, can be used interchangeably to mean that a cell (e.g., yeast cell) is maintained outside its normal environment under controlled conditions, e.g., under conditions suitable for survival. Cultured cells are allowed to survive, and culturing can result in cell growth, stasis, differentiation or division. The term does not imply that all cells in the culture survive, grow, or divide, as some may naturally die or senesce. Cells are typically cultured in media, which can be changed during the course of the culture.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

As used herein, the term “a stabilizing S′ sequence specific RNA cleavage site sequence” refers to a nucleic acid sequence 5′ to a retron that, upon expression as an RNA, can be cleaved from the retron RNA and leaves a stabilizing sequence on the remaining retron RNA. Non-limiting examples of a stabilizing sequence can be the cleavage product of the Hepatitis Delta Virus (HDV) ribozyme, a stabilizing stem loop structure, a stem loop with a highly stable tetraloop, such as a GNRA or UNCG tetraloop, or a pseudoknot.

As used herein, the term “a stabilizing S′ ribozyme sequence” refers to a nucleic acid sequence encoding a ribozyme located 5′ to a retron that, upon expression as an RNA, cleaves itself from the retron RNA and leaves a stabilizing sequence on the remaining retron RNA. Non-limiting examples of a stabilizing sequence can be the cleavage product of the Hepatitis Delta Virus (HDV) ribozyme, a stabilizing stem loop structure, a stem loop with a highly stable tetraloop, such as a GNRA or UNCG tetraloop, or a pseudoknot.

As used herein, the term “a stem loop-stabilizing 5′ ribozyme sequence” refers to a nucleic acid sequence encoding a ribozyme located 5′ to a retron that, upon expression as an RNA, cleaves itself from the retron RNA and leaves a stabilizing sequence such as a stabilizing stem loop structure. A non-limiting example is RiboJ.

As used herein, the term “a 3′ ribozyme sequence” refers to a nucleic acid sequence encoding a ribozyme located 3′ to a retron. Non-limiting examples of 3′ ribozyme sequences include a Hammerhead ribozyme (HHR), HDV, RiboJ, and CPEB3.

The term “ribozyme” refers to an RNA molecule that is capable of catalyzing a biochemical reaction. In some instances, ribozymes function in protein synthesis, catalyzing the linking of amino acids in the ribosome. In other instances, ribozymes participate in various other RNA processing functions, such as splicing, viral replication, and tRNA biosynthesis. In some instances, ribozymes can be self-cleaving. Non-limiting examples of ribozymes include the HDV ribozyme, the Lariat capping ribozyme (formally called GIRI branching ribozyme), the glmS ribozyme, group I and group II self-splicing introns, the hairpin ribozyme, the hammerhead ribozyme, various rRNA molecules, RNase P, the twister ribozyme, the VS ribozyme, the pistol ribozyme, and the hatchet ribozyme. Other examples include the self-cleaving ribozyme-containing R2 elements, the LITc retrotransposon found in Trypanosoma cruzi, short interspaced nuclear elements (SINEs) in Schistosomes, Penelope-like elements and retrozymes. For more information regarding ribozymes, see, e.g., Doherty, et al. Ann. Rev. Biophys. Biomol. Struct. 30:457-475 (2001) and Weinberg, et al., Nucleic Acids Research, (47) 18:9480-9494 (2019); incorporated herein by reference in its entirety for all purposes.

As used herein, the term “a structure-forming nucleic acid within the msd sequence” refers to an exogenous nucleic acid sequence inserted within the loop-forming structure of the msd sequence that is able to form a structured region of nucleic acid when expressed as a retron ncRNA. The exogenous nucleic acid sequence can be placed adjacent to the programmed ssDNA sequence (i.e., donor) in the same loop of the msd region. In the retron ncRNA form, the structure resides 3′ of the donor or programmed ssDNA sequence. In the retron msDNA form, this becomes the S′ end. This structure can also be placed on the other side of the programmed ssDNA sequence. While not wishing to be held by theory, this structure may aid the proper folding of the msr/msd structure in the retron ncRNA to enhance reverse transcription, or may enhance the stability of the msDNA and protect it from cellular nucleases.

“Percent similarity,” or “percent identity” in the context of polynucleotide or peptide sequences, is determined by comparing two optimally aligned sequences over a comparison window, wherein the portion of the sequence (e.g., an msr locus sequence) in the comparison window may comprise additions or deletions (i.e., gaps) as compared to the reference sequence which does not comprise additions or deletions, for optimal alignment of the two sequences. The percentage is calculated by determining the number of positions at which the identical nucleotide or amino acid occurs in both sequences to yield the number of matched positions, dividing the number of matched positions by the total number of positions in the window of comparison and multiplying the result by 100 to yield the percentage of similarity (e.g., sequence similarity).

Two sequence are said to be “substantially similar” or “substantially identical” when a polynucleotide or peptide has at least about 70% similarity (e.g., sequence similarity), preferably at least about, or greater than or equal to, 85%, 86%, 87%, 88%, 89%, 90%, 91%, 92%, 93%, 94%, 95%, 96%, 97%, 98%, 99%, or 100% similarity, to a reference sequence, when compared and aligned for maximum correspondence over a comparison window, or designated region as measured using one of the following sequence comparison algorithms or by manual alignment and visual inspection. With regard to polynucleotide sequences, this definition also refers to the complement of a test sequence.

Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement))

Additional examples of algorithms that are suitable for determining percent sequence similarity are the BLAST and BLAST 2.0 algorithms, which are described in Altschul et al., (1990) J. Mol. Biol. 215:403-410 and Altschul et al. (1977) Nucleic Acids Res. 25:3389-3402, respectively. Software for performing BLAST analyses is publicly available at the National Center for Biotechnology Information website, ncbi.nlm.nih.gov. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see, e.g., Henikoff and Henikoff, Proc. Natl. Acad. Sci. USA 89:10915 (1989)).

The BLAST algorithm also performs a statistical analysis of the similarity between two sequences (see, e.g., Karlin and Altschul, Proc. Natl. Acad. Sci. USA, 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

Another method of establishing percent identity in the context of the present disclosure is to use the MPSRCH package of programs copyrighted by the University of Edinburgh, developed by John F. Collins and Shane S. Sturrok, and distributed by IntelliGenetics, Inc. (Mountain View, CA). From this suite of packages, the Smith Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used with the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs are readily available.

The term “transfection” is used to refer to the uptake of foreign DNA by a cell. A cell has been “transfected” when exogenous nucleic acids have been introduced inside the cell membrane. A number of transfection techniques are generally known in the art. See, e.g., Graham et al. (1973) Virology, 52:456, Sambrook et al. (2001) Molecular Cloning, a laboratory manual, 3rd edition, Cold Spring Harbor Laboratories, New York, Davis et al. (1995) Basic Methods in Molecular Biology, 2nd edition, McGraw-Hill, and Chu et al. (1981) Gene 13:197. Such techniques can be used to introduce one or more exogenous nucleic acid moieties into suitable host cells. The term refers to both stable and transient uptake of the genetic material and includes uptake of peptide- or antibody-linked nucleic acids.

The term “donor polynucleotide” or “donor sequence” refers to a polynucleotide that provides a sequence of an intended edit to be integrated into the genome at a target locus by HDR.

As used herein, the term “administering” includes oral administration, topical contact, administration as a suppository, intravenous, intraperitoneal, intramuscular, intralesional, intrathecal, intranasal, or subcutaneous administration to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. Administering also refers to delivery of material, including biological material such as nucleic acids and/or proteins, into cells by any suitable method including transformation, transfection, transduction, ballistic methods and/or electroporation.

The term “treating” refers to an approach for obtaining beneficial or desired results including, but not limited to, a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested.

The term “effective amount” or “sufficient amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The specific amount may vary depending on one or more of: the particular agent chosen, the host cell type, the location of the host cell in the subject, the dosing regimen to be followed, whether it is administered in combination with other compounds, timing of administration, and the physical delivery system in which it is carried.

The term “pharmaceutically acceptable carrier” refers to a substance that aids the administration of an active agent to a cell, an organism, or a subject. “Pharmaceutically acceptable carrier” refers to a carrier or excipient that can be included in the present compositions and that causes no significant adverse toxicological effect on the patient. Non-limiting examples of pharmaceutically acceptable carrier include water, NaCl, normal saline solutions, lactated Ringer's, normal sucrose, normal glucose, cell culture media, and the like. One of skill in the art will recognize that other pharmaceutical carriers are useful in the present methods and compositions.

### II. Compositions

### Nucleic Acids

In an aspect, provided herein are nucleic acids comprising: (i) a first promoter sequence operably linked to a retron nucleic acid sequence comprising: (a) an msr sequence, (b) an msd sequence, (c) a donor sequence within the msd sequence, and (d) a first inverted repeat sequence and a second inverted repeat sequence flanking the retron sequences; and (ii) a second promoter sequence operably linked to a nucleic acid sequence encoding a guide RNA region.

In some embodiments, the first promoter sequence operably linked to a nucleic acid sequence encoding a retron msr-msd-donor sequence (referred to as the “retron sequence”) and the second promoter operably linked to the nucleic acid sequence encoding a guide RNA region are present on the same plasmid or expression vector. In some embodiments, the first promoter is located upstream (5′) of the first inverted repeat sequence, and the second promoter is located downstream (3′) of the second inverted repeat sequence. In some embodiments, the first promoter is located downstream (3′) of the nucleic acid sequence encoding the guide RNA region.

In some embodiments, the first and second promoter sequences are same. In some embodiments, the first and second promoter sequences are different. In some embodiments, the first and/or second promoters can be an RNA polymerase II (Pol II) promoter or an RNA polymerase III (Pol III) promoter. In a particular embodiment, the first promoter comprises an RNA Pol II promoter, and the second promoter comprises an RNA Pol III promoter. In some embodiments, the Pol II promoter is selected from the yeast GAL7, human cytomegalovirus (CMV), cytomegalovirus immediate-early enhancer/chicken β-actin (CAG), a hybrid form of the CAG promoter (CBh), or Elongation factor 1-alpha (EF1a) promoters. In some embodiments, the Pol III promoter is selected from a human U6 or yeast SNR52 promoter.

Guide RNA (gRNA) Molecules

The retron-guide RNA cassettes and retron donor DNA-guide molecules of the present disclosure comprise DNA sequences encoding retron donor and guide RNA (gRNA) and the transcribed and processed retron donor and gRNA molecules, respectively. The gRNAs for use in the CRISPR-retron system as disclosed herein typically include a crRNA sequence that is complementary to a target nucleic acid sequence and may include a scaffold sequence (e.g., tracrRNA) that interacts with a Cas nuclease (e.g., Cas9), a Cas9 nickase or a variant or fragment thereof, depending on the particular nuclease being used.

The gRNA can comprise any nucleic acid sequence having sufficient complementarity with a target polynucleotide sequence (e.g., target DNA sequence) to hybridize with the target sequence and direct sequence-specific binding of a nuclease to the target sequence. The gRNA may recognize a protospacer adjacent motif (PAM) sequence that may be near or adjacent to the target DNA sequence. The target DNA site may lie immediately 5′ of a PAM sequence, which is specific to the bacterial species of the Cas9 used. For instance, the PAM sequence of Streptococcus pyogenes-derived Cas9 is NGG; the PAM sequence of Neisseria meningitidis-derived Cas9 is NNNNGATT; the PAM sequence of Streptococcus thermophilus-derived Cas9 is NNAGAA; and the PAM sequence of Treponema denticola-derived Cas9 is NAAAAC. In some embodiments, the PAM sequence can be 5′-NGG, wherein N is any nucleotide; 5′-NRG, wherein N is any nucleotide and R is a purine; or 5′-NNGRR, wherein N is any nucleotide and R is a purine. For the S. pyogenes system, the selected target DNA sequence should immediately precede (i.e., be located 5′ of) a S′NGG PAM, wherein N is any nucleotide, such that the guide sequence of the DNA-targeting RNA (e.g., gRNA) base pairs with the opposite strand to mediate cleavage at about 3 base pairs upstream of the PAM sequence.

In some embodiments, the degree of complementarity between a guide sequence of the gRNA (i.e., crRNA sequence) and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). In some embodiments, a crRNA sequence is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some instances, a crRNA sequence is about 20 nucleotides in length. In other instances, a crRNA sequence is about 15 nucleotides in length. In other instances, a crRNA sequence is about 25 nucleotides in length.

The nucleotide sequence of a modified gRNA can be selected using any of the web-based software described above. Considerations for selecting a DNA-targeting RNA include the PAM sequence for the nuclease (e.g., Cas9 or Cas9 nickase) to be used, and strategies for minimizing off-target modifications. Tools, such as the CRISPR Design Tool, can provide sequences for preparing the gRNA, for assessing target modification efficiency, and/or assessing cleavage at off-target sites.

In some embodiments, the length of the gRNA molecule is about 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, or more nucleotides in length. In some instances, the length of the gRNA is about 100 nucleotides in length. In other instances, the gRNA is about 90 nucleotides in length. In other instances, the gRNA is about 110 nucleotides in length.

### Donor DNA Sequences

In one aspect, the present disclosure provides guide RNA-msr-msd cassettes comprising an msd with an embedded donor DNA sequence. In another aspect, the present disclosure provides guide-retron donor DNA molecules comprising guide RNA-msr-msd transcripts that comprise donor DNA sequence coding regions, the transcripts subsequently being reverse transcribed to yield msDNA that comprises a donor DNA sequence. The donor DNA sequence or sequences participate in homology-directed repair (HDR) of genetic loci of interest following cleavage of genomic DNA at the genetic locus or loci of interest (i.e., after a nuclease has been directed to cut at a specific genetic locus of interest, targeted by binding of gRNA to a target sequence). Thus, in some embodiments, the donor sequence comprises a template for homology-directed repair (HDR). For example, the donor sequence can comprise two sequences (homology arms) that are homologous to the target DNA, one sequence located upstream of the target site and the other sequence located downstream of the target site.

In some embodiments, the recombinant donor repair template (i.e., donor DNA sequence) comprises two homology arms that are homologous to portions of the sequence of the genetic locus of interest at either side of a Cas nuclease (e.g., Cas9 or Cas9 nickase) cleavage site. The homology arms may be the same length or may have different lengths. In some instances, each homology arm has at least about 70 to about 99 percent similarity (i.e., at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent similarity) to a portion of the sequence of the genetic locus of interest at either side of a nuclease (e.g., Cas nuclease) cleavage site. In other embodiments, the recombinant donor repair template comprises or further comprises a reporter unit that includes a nucleotide sequence encoding a reporter polypeptide (e.g., a detectable polypeptide, fluorescent polypeptide, or a selectable marker). If present, the two homology arms can flank the reporter cassette and are homologous to portions of the genetic locus of interest at either side of the Cas nuclease cleavage site. The reporter unit can further comprise a sequence encoding a self-cleavage peptide, one or more nuclear localization signals, and/or a fluorescent polypeptide (e.g., enhanced green fluorescent protein (EGFP) or superfolder GFP (sfGFP)). Other suitable reporters are described herein.

In some embodiments, the donor DNA sequence is at least about 500 to 10,000 (i.e., at least about 500, 600, 700, 800, 900, 1,000, 1,100, 1,200, 1,300, 1,400, 1,500, 1,600, 1,700, 1,800, 1,900, 2,000, 2,500, 3,000, 3,500, 4,000, 4,500, 5,000, 5,500, 6,000, 6,500, 7,000, 7,500, 8,000, 8,500, 9,000, 9,500, or 10,000) nucleotides in length. In some embodiments, the donor DNA sequence is between about 600 and 1,000 (i.e., about 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1,000) nucleotides in length. In some embodiments, the donor DNA sequence is between about 100 and 500 (i.e., about 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500) nucleotides in length. In some embodiments, the donor DNA sequence is less than about 100 (i.e., less than about 100, 95, 90, 85, 80, 75, 70, 65, 60, 55, 50, 45, 40, 35, 30, 25, 20, 15, 10, or 5) nucleotides in length.

### Additional Components

The nucleic acid can comprise additional sequences located 5′ of the first inverted repeat and/or located 3′ of the second inverted repeat that stabilize the msr-msd-donor transcript (referred to as the retron transcript) in cells. For example, in some embodiments, the nucleic acid comprises a ribozyme sequence located 5′ of the first inverted repeat and/or 3′ of the second inverted repeat. In some embodiments, the nucleic acid comprises a 5′ ribozyme sequence located 3′ of the first promoter and 5′ of the first inverted repeat sequence. In some embodiments, the nucleic acid comprises a 3′ ribozyme sequence located 3′ of the second inverted repeat sequence.

Any S′ ribozyme can be used as long as it leaves a stabilizing sequence or pseudoknot when cleaved from the retron. The 5′ ribozyme sequence or sequences can be a Hammerhead Ribozyme (HHR), HDV ribozyme, RiboJ, CPEB3, Agam_1_1, Agam_2_2, Pmar 1, Bflo_1, Bflo_2, Spur_1, Spur_2, Spur_3, Spur_4, Ppac_1, Cjap_1, Fpra_1, CIV_1, Dpap_1, Tatr_1, CPEB3, G_HDV, A_HDV, Canis_familiaris/1/3 73, Felis catus_domestic_cat/1/3 74, Ailuropoda melanoleuca_Giant_p/3 73, Elephant/113/4 75, PongoAbelii_SumatranOrangutan//1 66, MicrocebusMurinus MouseLemur/1/1 66, TupaiaBelangeri_NorthernTreesh/1 66, Rabbit/84/4 75, Human. Chr10/290/4 75, Chimp_PanTroglodytes/49/4 75, Rhesus/23/4 75, Macaca_mulatta/1/1 70, SorexAraneus_CommonShrew/1/1 66, Mouse.chr19_CPEB3/70/4 75, Rat.Chr1/411/4 74, EquusCaballus/1/1 69, Lama_pacos_Alpaca/1/1 70, Opossum/55/4 75, Macropus_eugenii_Tammar_Wallab/1 72, Monodelphis_domestica_Grey_Sho/1 71, CaviaPorcellus GuineaPig/1/1 66, OchotonaPrinceps AmericanPike//1 66, Dasypus_novemcinctus_Nine Band/2 73, Choloepus_hoffmanni Hofmanns t/4 75, MyotisLucifugus_BrownBat/1/1 60, Cow/112/4 75, Callithrix jacchus Common marm/1 69, Tursiops_truncatus_Bottlenose/1 70, EchinopsTelfairi_HedgehogTenre/2 72, Sus Scrofa/1/2 71, Dipodomys_ordsii_Ords_Kangaroo/3 72, or Pteropus_vampyrus_Malayan Flyi/1 69, Gorilla_gorilla, self-cleaving ribozyme-containing R2 elements, the LITc retrotransposon found in Trypanosoma cruzi, short interspaced nuclear elements (SINEs) in Schistosomes, Penelope-like elements, and retrozymes. Examples of a 3′ ribozyme include, but are not limited to, Hammerhead ribozyme (HHR), HDV, RiboJ, or CPEB3. In some embodiments, nucleic acid encoding the retron transcript furthers comprises a stem-loop sequence located between the stabilizing 5′ ribozyme sequence and (a) the msr sequence.

In some embodiments, the nucleic acid comprises a long non-coding RNA (lncRNA) transcript located 3′ of the second inverted repeat sequence. In some embodiments, the lncRNA transcript comprises a Metastasis Associated Lung Adenocarcinoma Transcript 1 (MALATI).

In some embodiments, the nucleic acid comprises a transcription terminator sequence located 3′ of the second inverted repeat sequence and 5′ of the second promoter.

In some embodiments, the nucleic acids encoding the retron RNA and gRNA are introduced into a cell comprising a nucleic acid sequence encoding a reporter protein operably linked to a third promoter. In some embodiments, the third promoter is a Pol II promoter. In some embodiments, the reporter protein is a fluorescent protein, such as EGFP or dTomato.

### Retrons

Exemplary retrons comprising msr, msd, and inverted repeat sequences that can be used in the nucleic acids of the disclosure are provided in Table 1. The retrons in Table 1 also express reverse transcriptases that can be used in the methods of the disclosure. In some embodiments, the reverse transcriptase is encoded by a nucleic acid on a separate plasmid from the retron RNA.

TABLE 1

| | Name | Species | Bacterial taxon |
|---|---|---|---|
| 1 | Retron-Eco1 (Ec86) | Escherichia coli | Gammaproteobacteria |
| 2 | Retron-Eco2 (Ec67) | Escherichia coli | Gammaproteobacteria |
| 3 | Retron-Eco3 (Ec73) | Escherichia coli | Gammaproteobacteria |
| 4 | Retron-Eco4 (Ec83) | Escherichia coli | Gammaproteobacteria |
| 5 | Retron-Eco5 (Ec107) | Escherichia coli | Gammaproteobacteria |
| 6 | Retron-Eco6 (Ec48) | Escherichia coli | Gammaproteobacteria |
| 7 | Retron-Eco7 (Ec78) | Escherichia coli | Gammaproteobacteria |
| 8 | Retron-Eco9 | Escherichia coli | Gammaproteobacteria |
| 9 | Retron-Eco10 | Escherichia coli | Gammaproteobacteria |
| 10 | Retron-Eco11 | Escherichia coli | Gammaproteobacteria |
| 11 | Retron-Mxa1 (Mx162) | Myxococcus xanthus | Deltaproteobacteria |
| 12 | Retron-Mxa2 (Mx65) | Myxococcus xanthus | Deltaproteobacteria |
| 13 | Retron-Sau1 (Sa163) | Stigmatella aurantica | Deltaproteobacteria |
| 14 | Retron-Nex1 (Ne160) | Nannocystis exedens | Deltaproteobacteria |
| 15 | Retron-Nex2 (Ne144) | Nannocystis exedens | Deltaproteobacteria |
| 16 | Retron-Sen1 (Se72) | Salmonella enterica | Gammaproteobacteria |
| | | Enteridis | |
| 17 | Retron-Sen2 (St85) | Salmonella enterica | Gammaproteobacteria |
| | | Typhimurium | |
| 18 | Retron-Vch1 (Vc95) | Vibrio cholerae | Gammaproteobacteria |
| 19 | Retron-Vch2 (Vc81) | Vibrio cholerae | Gammaproteobacteria |
| 20 | Retron-Vch3 (Vc137) | Vibrio cholerae | Gammaproteobacteria |
| 21 | Retron-Vpa1 (Vp96) | Vibrio parahaemolyticus | Gammaproteobacteria |
| 22 | Retron-Kpn1 | Klebsiella pneumoniae | Gammaproteobacteria |
| 23 | Retron-Pmi1 | Proteus mirabilis | Gammaproteobacteria |
| 24 | Retron-Rxx1 | Rhizobium sp. | Alphaproteobacteria |
| 25 | Retron-Bxx1 | Bradyrhizobium sp. | Alphaproteobacteria |
| 26 | Retron-Fel1 | Flexibacter elegans | Bacteriodes |
| 27 | Retron-Cco1 | Corallococcus coralloides | Deltaproteobacteria |
| 28 | Retron-Adi1 | Angiococcus disciformis | Deltaproteobacteria |
| 29 | Retron-Age1 | Archangium gephyra | Deltaproteobacteria |
| 30 | Retron-Cfe1 | Cystobacter ferrugineus | Deltaproteobacteria |
| 31 | Retron-Cfu1 | Cystobacter fuscus | Deltaproteobacteria |
| 32 | Retron-Cvi1 | Cystobacter violaceus | Deltaproteobacteria |
| 33 | Retron-Mli1 | Melittangium lichenicola | Deltaproteobacteria |
| 34 | Retron-Mco1 | Myxoccocus coralloides | Deltaproteobacteria |
| 35 | Retron-Mfu1 | Myxococcus fulvus | Deltaproteobacteria |
| 36 | Retron-Mma1 | Myxococcus macrosporus | Deltaproteobacteria |
| 37 | Retron-Mst1 | Myxococcus stipitatus | Deltaproteobacteria |
| 38 | Retron-Mvi1 | Myxococcus virescens | Deltaproteobacteria |
| 39 | Retron-Cap1 | Chondromyces apiculatus | Deltaproteobacteria |
| 40 | Retron-Cpe1 | Chondromyces pedicalatus | Deltaproteobacteria |
| 41 | Retron-Sce1 | Sorangium cellulosum | Deltaproteobacteria |

(see Simon, A. J., et al., Retrons and their applications in genome engineering, Nucleic Acids Research, Volume 47, Issue 21, 2 Dec. 2019, Pages 11007-11019).

In some embodiments, the retron encoded by the nucleic acids described herein is a Retron-Ecol (Ec73) retron. In some embodiments, the reverse transcriptase encoded by a nucleic acid on a separate plasmid described herein is RT-Ec73.

### Plasmids

Also provided are plasmids comprising a nucleic acid of the disclosure. In some embodiments, the plasmid comprises a nucleic acid sequence (e.g., a DNA sequence) comprising: (i) a first promoter sequence operably linked to a retron nucleic acid sequence comprising: (a) an msr sequence, (b) an msd sequence, (c) a donor sequence within the msd sequence, and (d) a first inverted repeat sequence and a second inverted repeat sequence flanking the retron sequences; and (ii) a second promoter sequence operably linked to a nucleic acid sequence encoding a guide RNA region. In some embodiments, the plasmid is an expression plasmid.

In some embodiments, the first promoter operably linked to the retron sequence is located upstream (5′) of the first inverted repeat sequence, and the second promoter operably linked to the nucleic acid sequence encoding a guide RNA region is located downstream (3′) of the second inverted repeat sequence on the plasmid. In some embodiments, the first promoter operably linked to the retron sequence is located downstream (3′) of the nucleic acid sequence encoding the guide RNA region.

In some embodiments, the first and second promoter sequences are same. In some embodiments, the first and second promoter sequences are different. In some embodiments, the first and/or second promoters can be an RNA polymerase II (Pol II) promoter or an RNA polymerase III (Pol III) promoter. In a particular embodiment, the first promoter comprises an RNA Pol II promoter, and the second promoter comprises an RNA Pol III promoter. In some embodiments, the Pol II promoter is selected from a GAL7, CMV, CAG, CBh or EF1a promoter. In some embodiments, the Pol III promoter is selected from a U6 or SNR52 promoter.

It will be understood that in some embodiments the plasmid comprises sequences comprising two separate transcription units, a first transcription unit that expresses the retron sequence and a second transcription unit that expresses a CRISPR/Cas guide RNA. Thus, in some embodiments, the transcription products of the separate (“split”) retron and guide transcription units are not linked, coupled or fused to each other. In some embodiments, the plasmid comprises sequences comprising a single transcription unit, wherein the retron sequence and the CRISPR/Cas guide RNA are transcribed as a single transcript (e.g., from the same promoter or transcription start site), and the retron and guide RNA sequences are then separated after transcription by an RNA processing enzyme such as a ribozyme or endonuclease cleavage.

The plasmid can comprise additional sequences located 5′ of the first inverted repeat and/or located 3′ of the second inverted repeat that stabilize the msr-msd-donor transcript (referred to as the retron transcript) in cells. For example, in some embodiments, the plasmid comprises a ribozyme sequence located S′ of the first inverted repeat and/or 3′ of the second inverted repeat. In some embodiments, the plasmid comprises a 5′ ribozyme sequence located 3′ of the first promoter and 5′ of the first inverted repeat sequence. In some embodiments, the plasmid comprises a 3′ ribozyme sequence located 3′ of the second inverted repeat sequence.

### III. Systems

Also provided are systems for introducing genetic modifications at a target DNA locus. The system can include an expression plasmid of the disclosure, and plasmids that can be used to express a CRISPR-associated endonuclease and/or a reverse transcriptase (RT). In some embodiments, the system comprises:
(a) a first expression plasmid comprising a nucleic acid sequence comprising: (i) a first promoter sequence operably linked to a nucleic acid sequence encoding a retron comprising: (a) an msr sequence, (b) an msd sequence, (c) a donor sequence within the msd sequence, and (d) a first inverted repeat sequence and a second inverted repeat sequence flanking the retron sequences; and (ii) a second promoter sequence operably linked to a nucleic acid sequence encoding a guide RNA region;
(b) a second expression plasmid comprising a nucleic acid sequence encoding a CRISPR-associated endonuclease; and
(c) a third expression plasmid comprising a nucleic acid sequence encoding a reverse transcriptase.

In some embodiments, the second and third expression plasmids comprise a promoter operably linked to the nucleic acid sequences encoding the CRISPR-associated endonuclease and the reverse transcriptase, respectively. In some embodiments, the promoter is an RNA Pol II promoter or an RNA Pol III promoter. In some embodiments, the second and third expression plasmids comprise a Pol II promoter operably linked to the nucleic acid sequences encoding the CRISPR-associated endonuclease and the reverse transcriptase, respectively.

In some embodiments, the CRISPR-associated endonuclease generates a single-stranded nick in one strand of the target DNA locus instead of a double-strand break (DSB). In some embodiments, the CRISPR-associated endonuclease comprises a nickase. In some embodiments, the nickase is selected from the group consisting of nCas9-D10A and nCas9-H840A. In some embodiments, the nickase is nCas9-H840A.

In some embodiments, the reverse transcriptase is selected from the group consisting of RT-Ec73, RT-St85, RT-Ec83, RT-Ec78, RT-Eco9, RT-Eco10, and RT-Eco11. In some embodiments, the reverse transcriptase is RT-Ec73.

### IV. Methods of Use

Also provided herein are methods of generating retron nucleic acid and gRNA in a cell by introducing into the cell any of the nucleic acid compositions described above comprising: (i) a first promoter sequence operably linked to a nucleic acid sequence encoding a retron comprising: (a) an msr sequence, (b) an msd sequence, (c) a donor sequence within the msd sequence, and (d) a first inverted repeat sequence and a second inverted repeat sequence flanking the retron sequences; and (ii) a second promoter sequence operably linked to a nucleic acid sequence encoding a guide RNA region, and a reverse transcriptase or a nucleic acid encoding the same. In some embodiments, the donor sequence comprises sequences for HDR. In some embodiments, the nucleic acid further comprises a 5′ and/or 3′ ribozyme sequence described above.

Also disclosed are methods for editing DNA at a target locus in a cell. In some aspects, the method comprises introducing a system of the disclosure into a cell. In some embodiments, the method comprises introducing a system comprising A) a first expression plasmid comprising a nucleic acid sequence comprising: (i) a first promoter sequence operably linked to a nucleic acid sequence encoding a retron comprising: (a) an msr sequence, (b) an msd sequence, (c) a donor sequence within the msd sequence, and (d) a first inverted repeat sequence and a second inverted repeat sequence flanking the retron sequences; and (ii) a second promoter sequence operably linked to a nucleic acid sequence encoding a guide RNA region; B) a second expression plasmid comprising a nucleic acid sequence encoding a CRISPR-associated endonuclease; and C) a third expression plasmid comprising a nucleic acid sequence encoding a reverse transcriptase, into a cell.

The nucleic acids and plasmids can be introduced into the cell using any method known in the art, including transformation, transfection, transduction, ballistic methods and/or electroporation. In some embodiments, cells are transfected by chemical, electroporation, or lipofection methods.

In some embodiments, the first expression plasmid comprises a nucleic acid sequence, wherein the first promoter operably linked to the retron sequence is located upstream (5′) of the first inverted repeat sequence, and the second promoter operably linked to the nucleic acid sequence encoding a guide RNA region is located downstream (3′) of the second inverted repeat sequence on the plasmid. In some embodiments, the first promoter operably linked to the retron sequence is located downstream (3′) of the nucleic acid sequence encoding the guide RNA region.

In some embodiments, the first and second promoter sequences are same. In some embodiments, the first and second promoter sequences are different. In some embodiments, the first and/or second promoters can be an RNA polymerase II (Pol II) promoter or an RNA polymerase III (Pol III) promoter. In some embodiments, the first promoter comprises an RNA polymerase II (Pol II) promoter, and the second promoter comprises an RNA polymerase III (Pol III) promoter. In some embodiments, the Pol II promoter is selected from a GAL7, CMV, CAG, CBh or EF1a promoter. In some embodiments, the Pol III promoter is selected from a U6 or SNR52 promoter.

In some embodiments, the first expression plasmid contains sequences comprising two separate transcription units, a first transcription unit that expresses the retron sequence and a second transcription unit that expresses a CRISPR/Cas guide RNA. Thus, in some embodiments, the transcription products of the separate (“split”) retron and guide transcription units are not linked, coupled or fused to each other.

The plasmid can comprise additional sequences located 5′ of the first inverted repeat and/or located 3′ of the second inverted repeat that stabilize the msr-msd-donor transcript (referred to as the retron transcript) in cells. For example, in some embodiments, the plasmid comprises a ribozyme sequence located 5′ of the first inverted repeat and/or 3′ of the second inverted repeat. In some embodiments, the plasmid comprises a 5′ ribozyme sequence located 3′ of the first promoter and 5′ of the first inverted repeat sequence. In some embodiments, the plasmid comprises a 3′ ribozyme sequence located 3′ of the second inverted repeat sequence.

In some embodiments, the second and third expression plasmids comprise a promoter operably linked to the nucleic acid sequences encoding the CRISPR-associated endonuclease and the reverse transcriptase, respectively. In some embodiments, the promoter is an RNA Pol II promoter or an RNA Pol III promoter. In some embodiments, the second and third expression plasmids comprise an RNA Pol II promoter operably linked to the nucleic acid sequences encoding the CRISPR-associated endonuclease and the reverse transcriptase, respectively.

In some embodiments, the reverse transcriptase is selected from the group consisting of RT-Ec73, RT-St85, RT-Ec83, RT-Ec78, RT-Eco9, RT-Eco10, and RT-Eco11. In some embodiments, the reverse transcriptase is RT-Ec73.

In some embodiments, the CRISPR-associated endonuclease generates a single-stranded nick in one strand of the target DNA locus instead of a double-strand break (DSB). In some embodiments, the CRISPR-associated endonuclease comprises a nickase. In some embodiments, the nickase is selected from the group consisting of nCas9-D10A and nCas9-H840A. In some embodiments, the nickase is nCas9-H840A.

In some embodiments of the method, the editing efficiency of the split system (i.e., the retron and guide RNAs are not physically coupled) at the target locus is increased compared to a fusion system comprising a first expression plasmid wherein transcription products of the retron and the gRNA coding region are physically coupled. In some embodiments, the editing efficiency of the split system at the target locus is increased compared to a fusion system comprising a first expression plasmid wherein the retron msr-msd-donor RNA sequence is fused to the 3′ end of the gRNA. In some embodiments, the editing efficiency at the target locus is increased when the CRISPR-associated endonuclease is a nickase compared to a system wherein the CRISPR-associated endonuclease is wild-type Cas9. In some embodiments, the editing efficiency of the split system at the target locus is increased compared to a fusion system comprising both i) a first expression plasmid wherein transcription products of the retron and the gRNA coding region are physically coupled, and ii) a second expression plasmid comprising a nucleic acid sequence encoding a nickase compared to a wild-type Cas9. In some embodiments, the nickase is nCas9-D10A or nCas9-H840A.

In some embodiments, fusion of the guide RNA to the retron msr-msd-donor is not required for editing the target locus in a cell when the CRISPR-associated endonuclease is a nickase.

In some embodiments, the methods result in efficient editing at a target DNA locus and also minimize formation of indels and other undesirable on-target and off-target effects.

In some embodiments, the cell is a eukaryotic cell. In some embodiments, the cell is selected from the group consisting of a yeast cell, mammalian cell, mammalian cell line, human cell, and human cell line.

### V. Methods for Preventing or Treating Genetic Diseases

Also provided herein are methods of treating a genetic disease in a subject in need thereof, comprising administering to the subject (a) any of the nucleic acid compositions described above comprising (i) a first promoter sequence operably linked to a nucleic acid sequence encoding a retron comprising: (a) an msr sequence, (b) an msd sequence, (c) a donor sequence within the msd sequence, and (d) a first inverted repeat sequence and a second inverted repeat sequence flanking the retron sequences; and (ii) a second promoter sequence operably linked to a nucleic acid sequence encoding a guide RNA region, (b) a CRISPR-associated endonuclease or a nucleic acid sequence encoding same, and (c) a reverse transcriptase or a nucleic acid sequence encoding same.

In another aspect, the present disclosure provides a pharmaceutical composition comprising: (a) a nucleic acid of the disclosure, a plasmid of the disclosure, a system of the disclosure, or a combination thereof, and (b) a pharmaceutically acceptable carrier. In some embodiments, the pharmaceutical composition comprises a nucleic acid or a plasmid comprising (i) a first promoter sequence operably linked to a nucleic acid sequence encoding a retron comprising: (a) an msr sequence, (b) an msd sequence, (c) a donor sequence within the msd sequence, and (d) a first inverted repeat sequence and a second inverted repeat sequence flanking the retron sequences; and (ii) a second promoter sequence operably linked to a nucleic acid sequence encoding a guide RNA region. In some embodiments, the pharmaceutical composition further comprises a CRISPR-associated endonuclease or a nucleic acid sequence encoding same, and a reverse transcriptase or a nucleic acid sequence encoding same.

In any of the embodiments described herein, the CRISPR-associated endonuclease is selected from a wild-type Cas9, a nickase, or modified variant thereof. In some embodiments, the Cas9 nuclease is a modified variant that does not catalyze site-directed cleavage of DNA to generate double-strand breaks. In some embodiments, the Cas9 variant is selected from nCas9-D10A and nCas9-H840A. In some embodiments, the Cas9 variant is nCas9-H840A.

In some embodiments, the reverse transcriptase is selected from the group consisting of RT-Ec73, RT-St85, RT-Ec83, RT-Ec78, RT-Eco9, RT-Eco10, and RT-Eco11. In some embodiments, the reverse transcriptase is RT-Ec73.

In yet another aspect, provided herein is a method for preventing or treating a genetic disease in a subject, the method comprising administering to the subject an effective amount of a pharmaceutical composition of the present disclosure to correct a mutation in a target gene associated with the genetic disease.

Genome editing may be performed on a single cell or a population of cells of interest and can be performed on any type of cell, including any cell from a prokaryotic, eukaryotic, or archaeon organism, including bacteria, archaea, fungi, protists, plants, and animals. Cells from tissues, organs, and biopsies, as well as recombinant cells, genetically modified cells, cells from cell lines cultured in vitro, and artificial cells (e.g., nanoparticles, liposomes, polymersomes, or microcapsules encapsulating nucleic acids) may all be used in the practice of the present disclosure. The methods of the disclosure are also applicable to editing of nucleic acids in cellular fragments, cell components, or organelles comprising nucleic acids (e.g., mitochondria in animal and plant cells, plastids (e.g., chloroplasts) in plant cells and algae). Cells may be cultured or expanded prior to or after performing genome editing as described herein. In one embodiment, the cells are yeast cells. In another embodiment, the cells are mammalian cells.

An RNA-guided nuclease can be targeted to a particular genomic sequence (i.e., genomic target sequence to be modified) by altering its guide RNA sequence. A target-specific guide RNA comprises a nucleotide sequence that is complementary to a genomic target sequence, and thereby mediates binding of the nuclease-gRNA complex by hybridization at the target site. For example, the gRNA can be designed with a sequence complementary to the sequence of a minor allele to target the nuclease-gRNA complex to the site of a mutation. The mutation may comprise an insertion, a deletion, or a substitution. For example, the mutation may include a single nucleotide variation, gene fusion, translocation, inversion, duplication, frameshift, missense, nonsense, or other mutation associated with a phenotype or disease of interest. The targeted minor allele may be a common genetic variant or a rare genetic variant. In certain embodiments, the gRNA is designed to selectively bind to a minor allele with single base-pair discrimination, for example, to allow binding of the nuclease-gRNA complex to a single nucleotide polymorphism (SNP). In particular, the gRNA may be designed to target disease-relevant mutations of interest for the purpose of genome editing to remove the mutation from a gene. Alternatively, the gRNA can be designed with a sequence complementary to the sequence of a major or wild-type allele to target the nuclease-gRNA complex to the allele for the purpose of genome editing to introduces a mutation into a gene in the genomic DNA of the cell, such as an insertion, deletion, or substitution. Such genetically modified cells can be used, for example, to alter phenotype, confer new properties, or produce disease models for drug screening.

In some embodiments, the RNA-guided nuclease used for genome modification is a clustered regularly interspaced short palindromic repeats (CRISPR) system Cas nuclease. In some embodiments, the Cas nuclease is a Cas9 nuclease or modified variant thereof. In some embodiments, the Cas nuclease is a Cas9 nuclease or modified variant thereof that does not catalyze site-directed cleavage of DNA to generate double-strand breaks. In some embodiments, the Cas nuclease is a Cas9 nickase, such as nCa9-D10A of nCas9-H840A.

The genomic target site will typically comprise a nucleotide sequence that is complementary to the gRNA and may further comprise a protospacer adjacent motif (PAM). In certain embodiments, the target site comprises 20-30 base pairs in addition to a 3 base pair PAM. Typically, the first nucleotide of a PAM can be any nucleotide, while the two other nucleotides will depend on the specific Cas9 protein that is chosen. Exemplary PAM sequences are known to those of skill in the art and include, without limitation, NNG, NGN, NAG, and NGG, wherein N represents any nucleotide. In certain embodiments, the allele targeted by a gRNA comprises a mutation that creates a PAM within the allele, wherein the PAM promotes binding of the Cas9-gRNA complex to the allele.

In certain embodiments, the gRNA is 5-50 nucleotides, 10-30 nucleotides, 15-25 nucleotides, 18-22 nucleotides, or 19-21 nucleotides in length, or any length between the stated ranges, including, for example, 10, 11, 12, 13, 14, 15, 16, 17, 18, 19, 20, 21, 22, 23, 24, 25, 26, 27, 28, 29, 30, 31, 32, 33, 34, or 35 nucleotides in length. The guide RNA may be a single guide RNA comprising erRNA and tracrRNA sequences in a single RNA molecule, or the guide RNA may comprise two RNA molecules with crRNA and tracrRNA sequences residing in separate RNA molecules.

The compositions and methods of the present disclosure are suitable for any disease that has a genetic basis and is amenable to prevention or amelioration of disease-associated sequelae or symptoms by editing or correcting one or more genetic loci that are linked to the disease. Non-limiting examples of diseases include X-linked severe combined immune deficiency, sickle cell anemia, thalassemia, hemophilia, neoplasia, cancer, age-related macular degeneration, schizophrenia, trinucleotide repeat disorders, fragile X syndrome, prion-related disorders, amyotrophic lateral sclerosis, drug addiction, autism, Alzheimer's disease, Parkinson's disease, cystic fibrosis, blood and coagulation diseases and disorders, inflammation, immune-related diseases and disorders, metabolic diseases and disorders, liver diseases and disorders, kidney diseases and disorders, muscular/skeletal diseases and disorders, neurological and neuronal diseases and disorders, cardiovascular diseases and disorders, pulmonary diseases and disorders, and ocular diseases. The compositions and methods of the present disclosure can also be used to prevent or treat any combination of suitable genetic diseases.

In some embodiments, the subject is treated before any symptoms or sequelae of the genetic disease develop. In other embodiments, the subject has symptoms or sequelae of the genetic disease. In some instances, treatment results in a reduction or elimination of the symptoms or sequelae of the genetic disease.

In some embodiments, treatment includes administering the herein-disclosed compositions directly to a subject. As a non-limiting example, pharmaceutical compositions as described herein can be delivered directly to a subject (e.g., by local injection or systemic administration). In other embodiments, the compositions are delivered to a host cell or population of host cells, and then the host cell or population of host cells is administered or transplanted to the subject. The host cell or population of host cells can be administered or transplanted with a pharmaceutically acceptable carrier. In some instances, editing of the host cell genome has not yet been completed prior to administration or transplantation to the subject. In other instances, editing of the host cell genome has been completed when administration or transplantation occurs. In certain instances, progeny of the host cell or population of host cells are transplanted into the subject. In some embodiments, correct editing of the host cell or population of host cells, or the progeny thereof, is verified before administering or transplanting edited cells or the progeny thereof into a subject. Procedures for transplantation, administration, and verification of correct genome editing are discussed herein and will be known to one of skill in the art.

Compositions of the present disclosure, including cells and/or progeny thereof that have had their genomes edited by the present methods and/or compositions, may be administered as a single dose or as multiple doses, for example two doses administered at an interval of about one month, about two months, about three months, about six months or about 12 months. Other suitable dosage schedules can be determined by a medical practitioner.

Prevention or treatment can further comprise administering agents and/or performing procedures to prevent or treat concomitant or related conditions. As non-limiting examples, it may be necessary to administer drugs to suppress immune rejection of transplanted cells, or prevent or reduce inflammation or infection. A medical professional will readily be able to determine the appropriate concomitant therapies.

### G. Kits

In another aspect, the present disclosure provides kits for modifying one or more target nucleic acids of interest at one or more target loci within a genome of a host cell, the kit comprising one or a plurality of nucleic acids, plasmids, or systems of the present disclosure. The kit may further comprise a host cell or a plurality of host cells.

In some embodiments, the kit contains one or more reagents. In some instances, the reagents are useful for transforming a host cell with a vector or a plurality of vectors, and/or inducing expression from the vector or plurality of vectors. In other embodiments, the kit may further comprise a reverse transcriptase, a plasmid for expressing a reverse transcriptase, one or more nucleases, one or more plasmids for expressing one or more nucleases, or a combination thereof. The kit may further comprise one or more reagents useful for delivering nucleases or reverse transcriptases into the host cell and/or inducing expression of the reverse transcriptase and/or the one or more nucleases. In yet other embodiments, the kit further comprises instructions for transforming the host cell with the vector, introducing nucleases and/or reverse transcriptases into the host cell, inducing expression of the vector, reverse transcriptase, and/or nucleases, or a combination thereof.

In yet another aspect, the present disclosure provides a kit for modifying one or more target nucleic acids of interest at one or more target loci within a genome of a host cell, the kit comprising one or a plurality of nucleic acid molecules, plasmids, or systems of the present disclosure. The kit may further comprise a host cell or a plurality of host cells.

In some embodiments, the kit contains one or more reagents. In some instances, the reagents are useful for introducing one or more of the nucleic acids or plasmids into the host coll. The kit may further comprise one or more reagents useful for inducing expression of any of the herein-described nucleic acids. In yet other embodiments, the kit further comprises instructions for introducing one or more of the nucleic acids or plasmids, for inducing expression of the separate gRNA and retron transcripts, or expression of the Cas9 or RT proteins, or a combination thereof.

### H. Applications

The compositions and methods provided by the present disclosure are useful for any number of applications. As non-limiting examples, genome editing can be performed to correct detrimental lesions in order to prevent or treat a disease, or to identify one or more specific genetic loci that contribute to a phenotype, disease, biological function, and the like. As another non-limiting example, genome editing or screening according to the compositions and methods of the present disclosure can be used to improve or optimize a biological function, pathway, or biochemical entity (e.g., protein optimization). Such optimization applications are especially suited to the compositions and methods of the present disclosure, as they can require the modification of a large number of genetic loci and subsequently assessing the effects.

Other non-limiting examples of applications suitable for the herein-disclosed compositions and methods include the production of recombinant proteins for pharmaceutical and industrial use, the production of various pharmaceutical and industrial chemicals, the production of vaccines and viral particles, and the production of fuels and nutraceuticals. All of these applications typically involve high-throughput or high-content screening, making them especially suited to the compositions and methods described herein.

In some embodiments, inducing one or more sequence modifications at one or more genetic loci of interest comprises substituting, inserting, and/or deleting one or more nucleotides at the one or more genetic loci of interest. In some instances, inducing the one or more sequence modifications results in the insertion of one or more sequences encoding cellular localization tags, one or more synthetic response elements, and/or one or more sequences encoding degrons into the genome.

In other embodiments, inducing the one or more sequence modifications at the one or more genetic loci of interest results in the insertion of one or more sequences from a heterologous genome. Introducing heterologous DNA sequences into a genome is useful for any number of applications, some of which are described herein. Others will be readily apparent to one of skill in the art. Non-limiting examples are directed protein evolution, biological pathway optimization, and production of recombinant pharmaceuticals.

### EXAMPLES

### Example 1

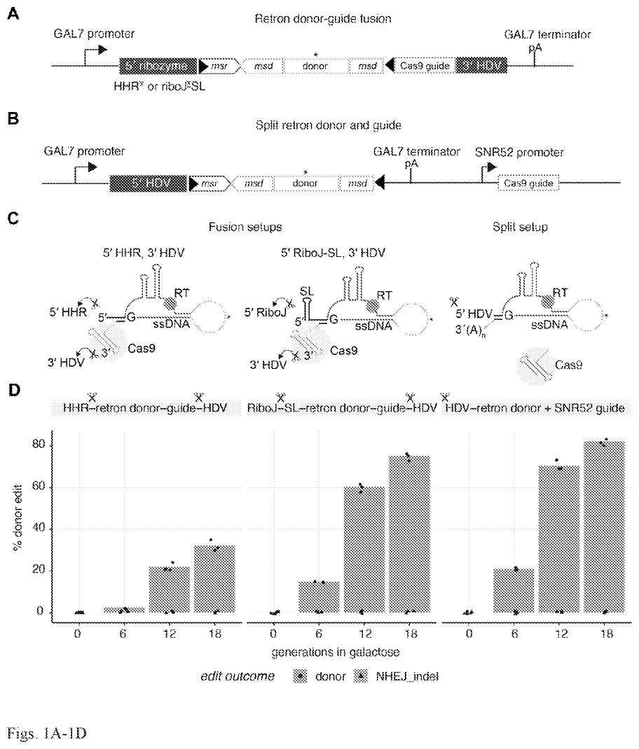

We have developed a more efficient retron editing system where the gRNA and msr-msd-donor are expressed from separate promoters (FIG. 1B, 2B). First, we demonstrate that in both yeast and mammalian cells, our split system is more efficient than the previously published fusion approaches, contradicting the model that retron donor must be recruited by Cas9 for efficient HDR. Second, we show that the split approach enables superior editing with Cas9-H840A nickase (nCas9-H840A), bypassing the levels observed with fully active Cas9 and retron-gRNA (rgRNA) fusions. By contrast, the rgRNA fusions lack detectable editing with nickases. The split retron-nickase approach holds great potential for numerous basic research and therapeutic applications, as it allows for precise editing while reducing formation of indels and other unwanted on-target and off-target effects.

### Technical Description:

Single-stranded donor DNA production by the retron system is achieved by inserting the sequence of the donor DNA within the msd part of the Pol-II RNA transcript (FIG. 1A). This yields an ssDNA part that can be leveraged to mediate HDR with combined Cas9-induced cleavage at the target site. In our earlier work, we had tested whether a previously published retron editing system employing a 5′-HHR-retron donor-guide-HDV-3′ system (Sharon et al., 2018) could be improved by altering the ribozymes at the 5′ and 3′ ends. This revealed that while the HDV ribozyme at the 3′ end is critical for editing, the 5′ RiboJ ribozyme vastly outperformed all other ribozymes tested at this 5′ position (U.S. provisional No. 63/214,197). Further dissection of the system revealed that the 3′ HDV, while critical for editing, severely reduced the levels of retron donor produced (U.S. provisional No. 63/214,197). On the other hand, the HDV ribozyme on the 5′ end dramatically boosted retron cDNA levels over the S′ RiboJ, but had exhibited reduced editing. We reconciled these disparate observations by reasoning that the retron donor and guide need different processing elements for maximum functionality, as the retron donor is likely not protected from cellular nucleases by the RT as the guide RNA is by Cas9. The RiboJ ribozyme leaves behind a stem-loop element which we reasoned provides some level of protection to the retron RNA (FIG. 1C). The fact that the guide should benefit from the removal of 5′ and 3′ extensions is consistent with previously published findings, motivating our efforts to separate the retron and guide for maximal guide efficacy (PMC4420636, PMC5686910). We therefore tested whether expressing the retron donor with the 5′ HDV and separately expressing the guide RNA from the well-characterized SNR52 non-coding RNA promoter would improve editing in budding yeast (U.S. provisional No. 63/214,197). Indeed, we observed enhanced editing at every time point for the split retron-guide system relative to the best fusion system, the 5′ RiboJ-retron donor-guide-HDV-3′ (FIG. 1D).

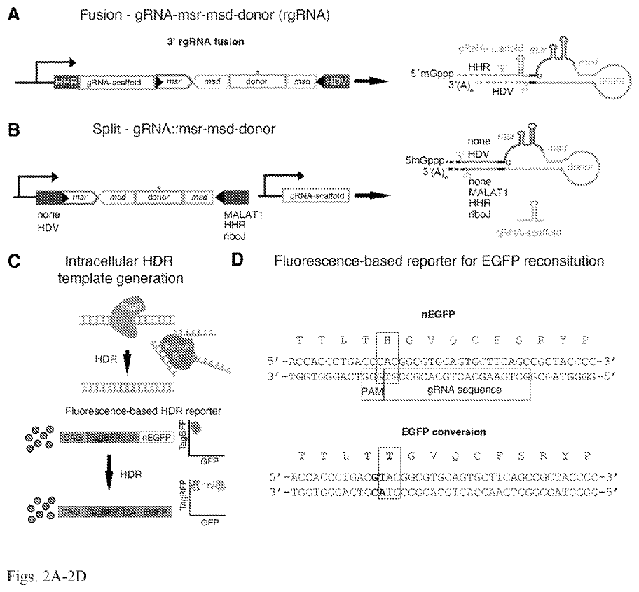

To test whether these findings would apply in human cells, we used a fluorescent readout based on EGFP reconstitution of a non-functional EGFP (nEGFP) to enable rapid assessment of HDR efficiencies. HEK 293 cells were infected with a lentivirus introducing CAG-TagBFP-P2A-nEGFP, where the mutation of a C->T result in an amino-acid change of a Histidine to a Tyrosine with subsequent EGFP fluorescence that can be measure by flow cytometry (FIGS. 2C and D). An additional silent mutation that disrupts the PAM was used in all HDR donor constructs to prevent recutting of the target site and digestion of the transfected plasmids.

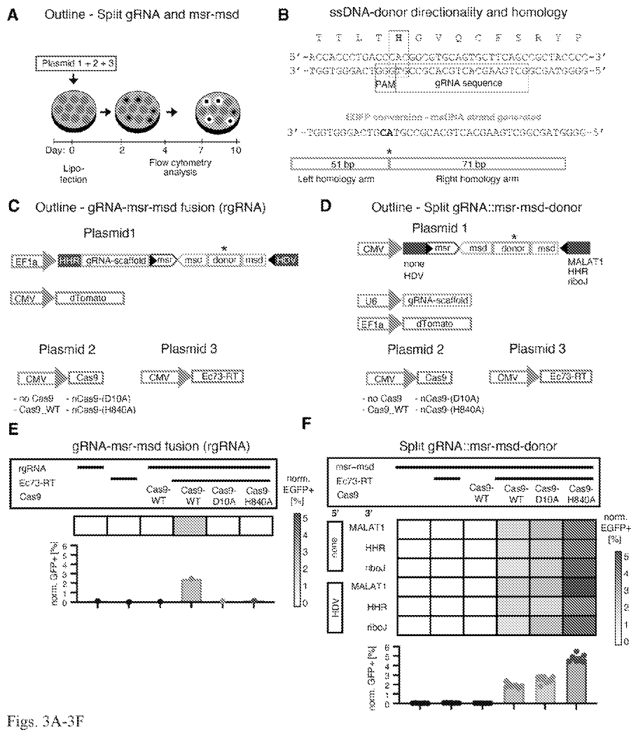

We tested the different constructs by lipofection of the relevant constructs in our reporter HEK293 cells (FIG. 3A). The retron donor/guide plasmids also harbor a dTomato fluorescent reporter as a control to indicate the fraction of cells that is transfected. As only these cells are able to receive an edit, the measured EGFP fluorescence was normalized to the percentage of dTomato positive cells at day 2. Transfection is typically an all-or-none process such that cells receiving plasmid 1 are likely to also receive plasmids 2 and 3, such that reporting on one of the plasmids is sufficient. The directionality of the generated msDNA-donor in our tests was the same strand as containing the PAM-NGG sequence (FIG. 3B), meaning that the guide RNA and retron donor DNA would bind to the same strand (top strand in FIG. 3B). The homology arms of the msDNA were 51 bp for the left arm and 71 bp for the right arm for all our constructs (FIG. 3B).

We first tested a modified version of the rgRNA system published by Kong et al. where the msr-msd-donor is fused to the 3′ end of the targeting gRNA (FIG. 3C). The Pol-II transcript is flanked by a HHR ribozyme at the 5′ end and an HDV ribozyme at the 3′ end. This plasmid also introduced the dTomato fluorescence used for normalization. A second plasmid introduces either double-strand break-inducing Cas9 or the nickase version nCas9-D10A or nCas9-H840A, that cleave only one DNA strand. A third plasmid introduces the Ec73 retron RT (RT-Ec73), crucial for reverse transcribing the msr-msd-donor. We then lipofected these plasmids into our reporter HEK293 cells (FIG. 3C). Controls with only rgRNA, Ec73 retron RT (RT-Ec73), or rgRNA combined with Cas9 did not result in GFP positive cells (FIG. 3E). Once all three constructs were introduced into cells, we observed 2.5% GFP positive cells using Cas9 after normalization, while we observed no detectable GFP positive cells above background using nCas9-D10A and nCas9-H840A.

Next we tested our split gRNA::msr-msd-donor approach. The first plasmid introduces the msr-msd-donor transcript, flanked by S′ and 3′ ribozymes that aim to make the transcript more stable in the cells, and a gRNA expressed from a separate Pol-III promoter. On the same plasmid dTomato expression reports on the percentage of transfected, which is used to normalize GFP positive cells. The second and third plasmids introducing Cas9 and the Ec73 retron are the same as outlined above. Plasmids were lipofected into our HEK293 reporter cells, and editing measured by flow cytometry. Double-strand break-inducing Cas9 showed editing comparable to the rgRNA fusions above ranging from 1.6-2.5% of GFP positive cells after normalization (FIG. 3F). nCas9-D10A showed slightly higher editing with a mean of 2.5% GFP positive cells, whereas nCas9-H840A showed a 2.5 fold increase in GFP positive cells compared to Cas9 with an average of 4.7% over the different 5′ and 3′ ribozyme conditions (FIG. 3F). Different ribozyme conditions showed only minimal effects on editing efficiency in our approach.

As we are testing only one directionality of msDNA-donor in this experiment, there is the possibility that by switching donor directionality we would observe enhanced editing efficiency using nCas9-D10A instead of nCas9-H840A, as they are nicking opposing strands respective to the gRNA directionality. nCas9-D10A and nCas9-H840A are reported to have drastically reduced on-target indel formation compared to Cas9 as well as reduced prevalence of off-target effects, which can be advantageous for a lot of applications. In our tests, the rgRNA fusions did show little editing with nCas9. In contrast, the split gRNA::msr-msd-donor nCas9-D10A showed comparable editing efficiencies to Cas9, while it was vastly increased using Cas9-H840A. Our results suggest that the fusion of gRNAs to the msr-msd-donor is not required to achieve editing, while the spit gRNA::msr-msd-donor approach allows to also achieve editing using nCas9-D10A and nCas9-H840A and at the same time minimize indel generation. Overall, these results are consistent with our findings in yeast that retrons and guides can function most efficiently when they are expressed separately with specific promoters and processing elements tailored to each.

### Example 2

We previously described that separately expressed CRISPR guide RNAs and retron donors work more efficiently in mediating homology directed repair (HDR) than reported retron-gRNA fusions. Furthermore, we showed that editing efficiency is increased using the Cas9 nickase H840A compared to the fully active Cas9 nuclease. Single-stranded nicks prevent the toxicity and error-prone repair pathways associated with double-strand breaks from fully active Cas9 and are therefore beneficial for potential therapeutic applications.

Retron-mediated generation of single-stranded donor DNA in mammalian HEK293 cells was achieved by expressing the Ec73 retron reverse transcriptase (RT-Ec73) and the corresponding msr-msd RNA transcript which comprises a distinct secondary structure that is crucial to initiate reverse transcription. We used a reporter system to measure editing efficiency whereby the retron single-stranded donor DNA (msDNA-donor) facilitates repair of a non-functional EGFP (nEGFP) construct that is targeted by Cas9 nuclease with either a nick or double-stranded DNA break. HDR efficiency was read out by measuring GFP positive cells via flow cytometry normalized to the percentage of total transfected cells.

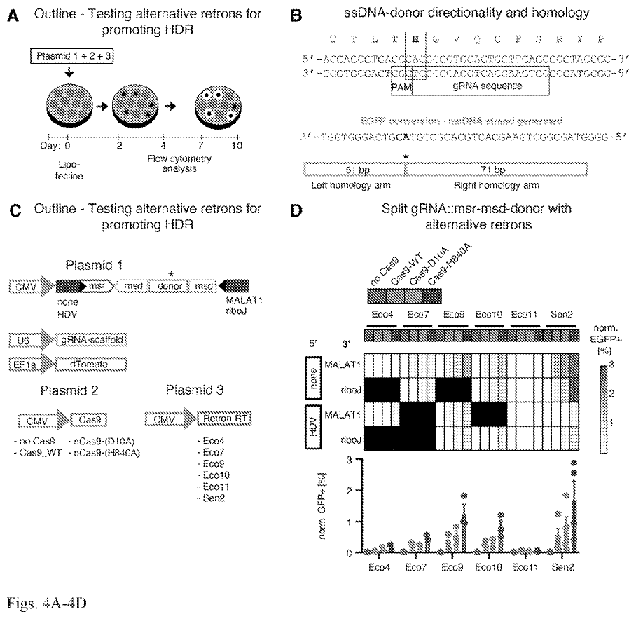

We tested whether HDR can also be achieved by expressing other retron RTs and their corresponding msr-msd-donor RNA transcripts. The retrons tested were Eco4, Eco7, Eco9, Eco10, Eco11 and Sen2. Testing was done by lipofecting the relevant plasmids into the HEK293 nEGFP reporter cell line (FIG. 4A). As previously described in Example 1, the retron donor/guide plasmids also express dTomato fluorescent protein that was used to normalize the measured fraction of EGFP positive cells over the total fraction of transfected cells. This plasmid was modified for each tested retron RT to contain the corresponding msr-msd-donor RNA transcript. The msDNA-donor was kept the same for all experiments and was kept constant to previous experiments using Ec73 (FIG. 4B). Three plasmids were introduced per lipofection (FIG. 4C) encoding for all the relevant parts to facilitate retron-mediated HDR in mammalian cells. Plasmid 1 introduces the msr-msd-donor RNA transcript flanked by different 5′ or 3′ stabilizing secondary RNA elements together with a gRNA targeting the nEGFP and a dTomato fluorescent protein. The retron msr-msd-donor and the gRNA are expressed as separate transcripts in this plasmid. Plasmid 2 encodes for different Cas9 nucleases that either facilitate a double-stranded break or a nick. Plasmid 3 expresses the different retron RTs. As shown in FIG. 4D, we observed varying HDR efficiencies for the different retrons that were tested, with the highest efficiency mediated by Sen2 (2.9%). This is lower than editing efficiencies that were measured using Ec73 in previous experiments (5.5%) as shown in FIG. 3F. Overall highest editing efficiencies with all retrons tested were achieved by using Cas9-H840A, confirming our previous finding that the split gRNA::msr-msd-donor system has highest editing efficiencies using this Cas9 nickase. In contrast to HDR mediated by Ec73, the addition of a 5′ HDV to the msr-msd-donor transcript yields a lower editing efficiency compared to the control transcripts which lacked a S′ stabilizing element or ribozyme. This indicates that the addition of 5′ or 3′ RNA processing and/or stability elements might have different effects on retron DNA output and/or HDR repair for different retron classes.

This example demonstrates that the split gRNA::msr-msd-donor system can also mediate HDR using alternative retron systems. Improvements on the msr-msd parts of the respective retrons, such as altering the lengths of stems or loops in the retron ncRNA, may result in improved performance of editing.

### REFERENCES

Farzadfard F, Lu T K. Synthetic biology. Genomically encoded analog memory with precise in vivo DNA writing in living cell populations. Science. 2014 Nov. 14; 346 (6211): 1256272. doi: 10.1126/science. 1256272. PMID: 25395541; PMCID: PMC4266475.
Kong X, Wang Z, Zhang R, Wang X, Zhou Y, Shi L, Yang H. Precise genome editing without exogenous donor DNA via retron editing system in human cells. Protein Cell. 2021 November; 12 (11): 899-902. doi: 10.1007/s13238-021-00862-7. Epub 2021 Aug. 17. PMID: 34403072; PMCID: PMC8563936.
Lopez S C, Crawford K D, Lear S K, Bhattarai-Kline S, Shipman S L. Precise genome editing across kingdoms of life using retron-derived DNA. Nat Chem Biol. 2022 February; 18 (2): 199-206. doi: 10.1038/s41589-021-00927-y. Epub 2021 Dec. 23. PMID: 34949838; PMCID: PMC8810715.
Sharon E, Chen S A, Khosla N M, Smith J D, Pritchard J K, Fraser H B. Functional Genetic Variants Revealed by Massively Parallel Precise Genome Editing. Cell. 2018 Oct. 4; 175 (2): 544-557.e16. doi: 10.1016/j.cell.2018.08.057. Epub 2018 Sep. 20. PMID: 30245013; PMCID: PMC6563827.
Simon A J, Ellington A D, Finkelstein I J. Retrons and their applications in genome engineering, Nucleic Acids Res. 2019 Dec. 2; 47 (21): 11007-11019. doi: 10.1093/nar/gkz865. PMID: 31598685; PMCID: PMC6868368.
Zhao B, Chen S A, Lee J, Fraser H B. Bacterial Retrons Enable Precise Gene Editing in Human Cells. CRISPR J. 2022 February; 5 (1): 31-39. doi: 10.1089/crispr.2021.0065. Epub 2022 Jan. 24. PMID: 35076284; PMCID: PMC8892976.

All publications and patent applications mentioned in this disclosure are herein incorporated by reference to the same extent as if each individual publication or patent application was specifically and individually indicated to be incorporated by reference.

No admission is made that any reference cited herein constitutes prior art. The discussion of the references states what their authors assert, and the Applicant reserves the right to challenge the accuracy and pertinence of the cited documents. It will be clearly understood that, although a number of information sources, including scientific journal articles, patent documents, and textbooks, may be referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure and are to be included within the scope of this application.

While particular alternatives of the present disclosure have been disclosed, it is to be understood that various modifications and combinations are possible and are contemplated within the scope of the appended claims. There is no intention, therefore, of limitations to the exact abstract and disclosure herein presented.

### EXEMPLARY EMBODIMENTS

Exemplary embodiments provided in accordance with the presently disclosed subject matter include, but are not limited to, the claims and the following embodiments:

Embodiment 1. A nucleic acid comprising: (i) a first promoter operably linked to a retron nucleic acid sequence comprising: an msr sequence; an msd sequence; a donor sequence within the msd sequence; and a first inverted repeat sequence and a second inverted repeat sequence; and (ii) a second promoter operably linked to a nucleic acid sequence encoding a guide RNA region.

Embodiment 2. The nucleic acid of embodiment 1, wherein the first promoter comprises an RNA polymerase II (Pol II) promoter, and the second promoter comprises an RNA polymerase III (Pol III) promoter.

Embodiment 3. The nucleic acid of embodiment 1 or 2, further comprising a stabilizing 5′ ribozyme sequence located 3′ of the first promoter and 5′ of the first inverted repeat sequence; a stabilizing 3′ ribozyme sequence located 3′ of the second inverted repeat sequence; and/or a long non-coding RNA (lncRNA) transcript located 3′ of the second inverted repeat sequence.

Embodiment 4. The nucleic acid of embodiment 3, wherein the stabilizing S′ or 3′ ribozyme sequence is selected from the group consisting of a hammerhead ribozyme (HHR), a hepatitis delta virus (HDV) ribozyme, and a RiboJ ribozyme sequence.

Embodiment 5. The nucleic acid of embodiment 3, wherein the stabilizing 3′ sequence comprises the 3′ triple-helix and tRNA-like processing components of the lncRNA transcript Metastasis Associated Lung Adenocarcinoma Transcript 1 (MALATI).

Embodiment 6. The nucleic acid of any one of embodiments 1 to 5, further comprising a transcription terminator sequence located 3′ of the second inverted repeat sequence and 5′ of the second promoter.

Embodiment 7. The nucleic acid of any one of embodiments 1 to 6, wherein the donor sequence comprises a template for homology-directed repair (HDR).

Embodiment 8. A nucleic acid comprising a transcription unit comprising a promoter operably linked to a retron nucleic acid sequence encoding a retron RNA and a nucleic acid sequence encoding a guide RNA region, wherein the retron nucleic acid sequence comprises: an msr sequence; an msd sequence; a donor sequence within the msd sequence; and a first inverted repeat sequence and a second inverted repeat sequence.

Embodiment 9. The nucleic acid of embodiment 8, wherein expression of the nucleic acid sequence results in a transcript comprising the retron RNA and guide RNA region, wherein the retron RNA and guide RNA region are separated after transcription by an RNA processing enzyme.

Embodiment 10. The nucleic acid of embodiment 9, wherein the RNA processing enzyme is a ribozyme or an endoribonuclease.

Embodiment 11. The nucleic acid of any one of embodiments 8 to 10, wherein the nucleic acid sequence encoding the retron RNA is located 5′ of the nucleic acid sequence encoding the guide RNA region.

Embodiment 12. The nucleic acid of any one of embodiments 8 to 11, wherein the promoter comprises an RNA polymerase II (Pol II) promoter or an RNA polymerase III (Pol III) promoter.

Embodiment 13. An expression plasmid comprising the nucleic acid of any one of embodiments 1 to 12.

Embodiment 14. A system for introducing a genetic modification at a target DNA locus, comprising: i) the expression plasmid of embodiment 13; ii) a second expression plasmid comprising a nucleic acid sequence encoding a CRISPR-associated endonuclease; and iii) a third expression plasmid comprising a nucleic acid sequence encoding a reverse transcriptase.

Embodiment 15. The system of embodiment 14, wherein the second and third expression plasmids comprise a promoter operably linked to the nucleic acid sequences encoding the CRISPR-associated endonuclease and the reverse transcriptase, respectively.

Embodiment 16. The system of embodiment 15, wherein the promoter is a Pol II promoter.

Embodiment 17. The system of any one of embodiments 14 to 16, wherein the CRISPR-associated endonuclease generates a single-stranded nick in one strand of the target DNA locus instead of a double-strand break (DSB).

Embodiment 18. The system of any one of embodiments 14 to 17, wherein CRISPR-associated endonuclease comprises a nickase selected from the group consisting of nCas9-D10A and nCas9-H840A.

Embodiment 19. The system of any one of embodiments 14 to 18, wherein the reverse transcriptase is selected from the group consisting of RT-Ec73, RT-St85, RT-Ec83, RT-Ec78, RT-Eco9, RT-Eco10, and RT-Eco11.

Embodiment 20. The system of any one of embodiments 14 to 19, wherein the reverse transcriptase is RT-Ec73.

Embodiment 21. A method for editing DNA at a target locus in a cell, comprising introducing the system of any one of embodiments 14 to 20 into the cell.

Embodiment 22. The method of embodiment 21, wherein the editing efficiency at the target locus is increased compared to a system comprising an expression plasmid wherein transcription products of the retron and the gRNA coding region are physically coupled.

Embodiment 23. The method of embodiment 21 or 22, wherein the cell is a eukaryotic cell.

Embodiment 24. The method of embodiment 23, wherein the cell is selected from the group consisting of a yeast cell, plant cell, mammalian cell, mammalian cell line, human cell, and human cell line.

Embodiment 25. A method of treating a genetic disease in a subject in need thereof, the method comprising administering to the subject an effective amount of a) the nucleic acid of any of embodiments 1 to 12, the plasmid of embodiment 13, the system of any one of embodiments 14 to 20, or a combination thereof; b) a reverse transcriptase or a nucleic acid encoding the same, and c) a sequence-specific endonuclease or a nucleic acid encoding the same.

Embodiment 26. The method of embodiment 25, wherein the reverse transcriptase is selected from the group consisting of RT-Ec73, RT-St85, RT-Ec83, RT-Ec78, RT-Eco9, RT-Eco10, and RT-Eco11.

Embodiment 27. The method of embodiment 25 or 26, wherein the reverse transcriptase is RT-Ec73.

Embodiment 28. The method of any one of embodiments 25 to 27, wherein the sequence-specific endonuclease does not catalyze a double strand break in the DNA of a host cell in the subject.

Embodiment 29. The method of any one of embodiments 25 to 28, wherein the sequence-specific endonuclease generates a single-stranded nick in one strand of a target DNA locus in a host cell of the subject.

Embodiment 30. The method of any one of embodiments 25 to 29, wherein the sequence-specific endonuclease is a nickase.

Embodiment 31. The method of embodiment 30, wherein the nickase is nCas9-D10A or nCas9-H840A.

Embodiment 32. The method of embodiment 31, wherein the nickase is nCas9-H840A.

Embodiment 33. A pharmaceutical composition comprising: (a) the nucleic acid of any one of embodiments 1 to 12, the plasmid of embodiment 13, or the system of any one of embodiments 14 to 20, or a combination thereof; and (b) a pharmaceutically acceptable carrier.

Embodiment 34. A method for preventing or treating a genetic disease in a subject, the method comprising administering to the subject an effective amount of the pharmaceutical composition of embodiment 33 to correct a mutation in a target gene associated with the genetic disease.

Embodiment 35. The method of any one of embodiments 25 to 32 or embodiment 34, wherein the genetic disease is selected from the group consisting of X-linked severe combined immune deficiency, sickle cell anemia, thalassemia, hemophilia, neoplasia, cancer, age-related macular degeneration, schizophrenia, trinucleotide repeat disorders, fragile X syndrome, prion-related disorders, amyotrophic lateral sclerosis, drug addiction, autism, Alzheimer's disease, Parkinson's disease, cystic fibrosis, blood and coagulation diseases and disorders, inflammation, immune-related diseases and disorders, metabolic diseases and disorders, liver diseases and disorders, kidney diseases and disorders, muscular/skeletal diseases and disorders, neurological and neuronal diseases and disorders, cardiovascular diseases and disorders, pulmonary diseases and disorders, ocular diseases and disorders, and a combination thereof.

Embodiment 36. A kit for modifying one or more target nucleic acids of interest at one or more target loci within a genome of a host cell, the kit comprising the nucleic acid of any of embodiments 1 to 12, or the plasmid of embodiment 13.

Embodiment 37. The kit of embodiment 36, further comprising a reverse transcriptase or a nucleic acid encoding the same, and a sequence-specific endonuclease or a nucleic acid encoding the same.

Embodiment 38. The kit of embodiment 37, wherein the reverse transcriptase is selected from the group consisting of RT-Ec73, RT-St85, RT-Ec83, RT-Ec78, RT-Eco9, RT-Eco10, and RT-Eco11.

Embodiment 39. The kit of embodiment 37 or 38, wherein the sequence-specific endonuclease comprises a nickase selected from the group consisting of nCas9-D10A and nCas9-H840A.

Embodiment 40. The kit of any one of embodiments 36 to 39, further comprising a host cell.

Embodiment 41. The kit of embodiment 40, wherein the host cell is a eukaryotic cell.

Embodiment 42. The kit of embodiment 40 or 41, wherein the host cell is selected from the group consisting of a yeast cell, a plant cell, mammalian cell, mammalian cell line, human cell, and human cell line.

Embodiment 43. The kit of any one of embodiments 36 to 42, further comprising one or more reagents for introducing the nucleic acids, plasmids, reverse transcriptase or sequence-specific endonuclease into the host cell.

Embodiment 44. A method for producing a retron RNA and a guide RNA, the method comprising: i) contacting the nucleic acid of any one of embodiments 8 to 12 with an RNA polymerase to produce a single transcript comprising the retron RNA and guide RNA sequences, and ii) contacting the single transcript with an RNA processing enzyme that cleaves the transcript between the retron RNA and guide RNA sequences, thereby producing the retron RNA and the guide RNA.

Embodiment 45. The method of embodiment 44, wherein the RNA processing enzyme is a ribozyme or an endoribonuclease.

Embodiment 46. The method of embodiment 44 or 45, wherein the nucleic acid sequence encoding the retron RNA is located 5′ of the nucleic acid sequence encoding the guide RNA.

INFORMAL SEQUENCE LISTING
Entire plasmid map for lentiviral construct encoding:
CAGG-TagBFP-T2A-nEGFP-WPRE
ttaatgtagtcttatgcaatactcttgtagtcttgcaacatggtaacgatgagttagcaaca
tgccttacaaggagagaaaaagcaccgtgcatgccgattggtggaagtaaggtggtacgatc
gtgccttattaggaaggcaacagacgggtctgacatggattggacgaaccactgaattgccg
cattgcagagatattgtatttaagtgcctagctcgatacataaacgggtctctctggttaga
ccagatctgagcctgggagctctctggctaactagggaacccactgcttaagcctcaataaa
gcttgccttgagtgcttcaagtagtgtgtgcccgtctgttgtgtgactctggtaactagaga
tccctcagacccttttagtcagtgtggaaaatctctagcagtggcgcccgaacagggacttg
aaagcgaaagggaaaccagaggagctctctcgacgcaggactcggcttgctgaagcgcgcac
ggcaagaggcgaggggcggcgactggtgagtacgccaaaaattttgactagcggaggctaga
aggagagagatgggtgcgagagcgtcagtattaagcgggggagaattagatcgcgatgggaa
aaaattcggttaaggccagggggaaagaaaaaatataaattaaaacatatagtatgggcaag
cagggagctagaacgattcgcagttaatcctggcctgttagaaacatcagaaggctgtagac
aaatactgggacagctacaaccatcccttcagacaggatcagaagaacttagatcattatat
aatacagtagcaaccctctattgtgtgcatcaaaggatagagataaaagacaccaaggaagc
tttagacaagatagaggaagagcaaaacaaaagtaagaccaccgcacagcaagcggccgctg
atcttcagacctggaggaggagatatgagggacaattggagaagtgaattatataaatataa
agtagtaaaaattgaaccattaggagtagcacccaccaaggcaaagagaagagtggtgcaga
gagaaaaaagagcagtgggaataggagctttgttccttgggttcttgggagcagcaggaagc
actatgggcgcagcgtcaatgacgctgacggtacaggccagacaattattgtctggtatagt
gcagcagcagaacaatttgctgagggctattgaggcgcaacagcatctgttgcaactcacag
tctggggcatcaagcagctccaggcaagaatcctggctgtggaaagatacctaaaggatcaa
cagctcctggggatttggggttgctctggaaaactcatttgcaccactgctgtgccttggaa
tgctagttggagtaataaatctctggaacagatttggaatcacacgacctggatggagtggg
acagagaaattaacaattacacaagcttaatacactccttaattgaagaatcgcaaaaccag
caagaaaagaatgaacaagaattattggaattagataaatgggcaagtttgtggaattggtt
taacataacaaattggctgtggtatataaaattattcataatgatagtaggaggcttggtag
gtttaagaatagtttttgctgtactttctatagtgaatagagttaggcagggatattcacca
ttatcgtttcagacccacctcccaaccccgaggggacccagaccacaattttaaaagaaaag
gggggattggggggtacagtgcaggggaaagaatagtagacataatagcaacagacatacaa
actaaagaattacaaaaacaaattacaaaaattcaaaattttcgggtttattacagggacag
cagagatccactttggcgccggctcgagtggctccggtgcccgtcagtGTCGACATTGATTA
TTGACTAGTTATTAATAGTAATCAATTACGGGGTCATTAGTTCATAGCCCATATATGGAGTT
CCGCGTTACATAACTTACGGTAAATGGCCCGCCTGGCTGACCGCCCAACGACCCCCGCCCAT
TGACGTCAATAATGACGTATGTTCCCATAGTAACGCCAATAGGGACTTTCCATTGACGTCAA
TGGGTGGACTATTTACGGTAAACTGCCCACTTGGCAGTACATCAAGTGTATCATATGCCAAG
TACGCCCCCTATTGACGTCAATGACGGTAAATGGCCCGCCTGGCATTATGCCCAGTACATGA
CCTTATGGGACTTTCCTACTTGGCAGTACATCTACGTATTAGTCATCGCTATTACCATGGGT
CGAGGTGAGCCCCACGTTCTGCTTCACTCTCCCCATCTCCCCCCCCTCCCCACCCCCAATTT
TGTATTTATTTATTTTTTAATTATTTTGTGCAGCGATGGGGGGGGGGGGGGGGGGGGCGCGC
GCCAGGCGGGGGGGGGGGGGGCGAGGGGCGGGGGGGGGCGAGGCGGAGAGGTGCGGCGGCAG
CCAATCAGAGCGGCGCGCTCCGAAAGTTTCCTTTTATGGCGAGGGGGGGGCGGCGGCGGCCC
TATAAAAAGCGAAGCGCGCGGCGGGCGGGAGTCGCTGCGTTGCCTTCGCCCCGTGCCCCGCT
CCGCGCCGCCTCGCGCCGCCCGCCCCGGCTCTGACTGACCGCGTTACTCCCACAGGTGAGCG
GGCGGGACGGCCCTTCTCCTCCGGGCTGTAATTAGCGCTTGGTTTAATGACGGCTCGTTTCT
TTTCTGTGGCTGCGTGAAAGCCTTAAAGGGCTCCGGGAGGGCCCTTTGTGCGGGGGGGAGCG
GCTCGGGGGGTGCGTGCGTGTGTGTGTGCGTGGGGAGCGCCGCGTGCGGCCCGCGCTGCCCG
GCGGCTGTGAGCGCTGCGGGCGCGGCGCGGGGCTTTGTGCGCTCCGCGTGTGCGCGAGGGGA
GCGCGGCCGGGGGGGGTGCCCCGCGGTGGGCGGGGGCTGCGAGGGGAACAAAGGCTGCGTGC
GGGGTGTGTGCGTGGGGGGGTGAGCAGGGGGTGTGGGCGCGGCGGTCGGGCTGTAACCCCCC
CCTGCACCCCCCTCCCCGAGTTGCTGAGCACGGCCCGGCTTCGGGTGCGGGGCTCCGTGCGG
GGCGTGGCGCGGGGCTCGCCGTGCCGGGCGGGGGGGGGCGGCAGGTGGGGGTGCCGGGCGGG
GCGGGGCCGCCTCGGGCCGGGGAGGGCTCGGGGGAGGGGCGCGGCGGCCCCGGAGCGCCGGC
GGCTGTCGAGGCGCGGCGAGCCGCAGCCATTGCCTTTTATGGTAATCGTGCGAGAGGGCGCA
GGGACTTCCTTTGTCCCAAATCTGGCGGAGCCGAAATCTGGGAGGCGCCGCCGCACCCCCTC
TAGCGGGCGCGGGCGAAGCGGTGCGGCGCCGGCAGGAAGGAAATGGGGGGGGAGGGCCTTCG
TGCGTCGCCGCGCCGCCGTCCCCTTCTCCATCTCCAGCCTCGGGGCTGCCGCAGGGGGACGG
CTGCCTTCGGGGGGGACGGGGCAGGGGGGGGTTCGGCTTCTGGCGTGTGACCGGCGGCTCTA
GAGCCTCTGCTAACCATGTTCATGCCTTCTTCTTTTTCCTACAGCTCCTGGGCAACGTGCTG
GTTATTGTGCTGTCTCATCATTTTGGCAAAGAATTCTGCAGTCGACGCCGCCACCATGAGCG
AGCTGATTAAGGAGAACATGCACATGAAGCTGTACATGGAGGGCACCGTGGACAACCATCAC
TTCAAGTGCACATCCGAGGGCGAAGGCAAGCCCTACGAGGGCACCCAGACCATGAGAATCAA
GGTGGTCGAGGGCGGCCCTCTCCCCTTCGCCTTCGACATCCTGGCTACTAGCTTCCTCTACG
GCAGCAAGACCTTCATCAACCACACCCAGGGCATCCCCGACTTCTTCAAGCAGTCCTTCCCT
GAGGGCTTCACATGGGAGAGAGTCACCACATACGAAGACGGGGGCGTGCTGACCGCTACCCA
GGACACCAGCCTCCAGGACGGCTGCCTCATCTACAACGTCAAGATCAGAGGGGTGAACTTCA
CATCCAACGGCCCIGTGATGCAGAAGAAAACACTCGGCIGGGAGGCCTTCACCGAGACGCTG
TACCCCGCTGACGGCGGCCTGGAAGGCAGAAACGACATGGCCCTGAAGCTCGTGGGCGGGAG
CCATCTGATCGCAAACATCAAGACCACATATAGATCCAAGAAACCCGCTAAGAACCTCAAGA
TGCCTGGCGTCTACTATGTGGACTACAGACTGGAAAGAATCAAGGAGGCCAACAACGAGACC
TACGTCGAGCAGCACGAGGTGGCAGTGGCCAGATACTGCGACCTCCCTAGCAAACTGGGGCA
CAAGCTTAATCCCGCTAGCGGATCGGGAGAGGGCAGAGGAAGTCTGCTAACATGCGGTGACG
TCGAGGAGAATCCTGGCCCACCGGTCGCCACCGTGAGCAAGGGCGAGGAGCTGTTCACCGGG
GTGGTGCCCATCCTGGTCGAGCTGGACGGCGACGTAAACGGCCACAAGTTCAGCGTGTCCGG
CGAGGGCGAGGGCGATGCCACCTACGGCAAGCTGACCCTGAAGTTCATCTGCACCACCGGCA
AGCTGCCCGTGCCCTGGCCCACCCTCGTGACCACCCTGACCCACGGCGTGCAGTGCTTCAGC
CGCTACCCCGACCACATGAAGCAGCACGACTTCTTCAAGTCCGCCATGCCCGAAGGCTACGT
CCAGGAGCGCACCATCTTCTTCAAGGACGACGGCAACTACAAGACCCGCGCCGAGGTGAAGT
TCGAGGGCGACACCCTGGTGAACCGCATCGAGCTGAAGGGCATCGACTTCAAGGAGGACGGC
AACATCCTGGGGCACAAGCTGGAGTACAACTACAACAGCCACAACGTCTATATCATGGCCGA
CAAGCAGAAGAACGGCATCAAGGTGAACTTCAAGATCCGCCACAACATCGAGGACGGCAGCG
TGCAGCTCGCCGACCACTACCAGCAGAACACCCCCATCGGCGACGGCCCCGTGCTGCTGCCC
GACAACCACTACCTGAGCACCCAGTCCGCCCTGAGCAAAGACCCCAACGAGAAGCGCGATCA
CATGGTCCTGCTGGAGTTCGTGACCGCCGCCGGGATCACTCTCGGCATGGACGAGCTGTACA
AGTAAtgaacgcgttaagtcgacaatcaacctctggattacaaaatttgtgaaagattgact
ggtattcttaactatgttgctccttttacgctatgtggatacgctgctttaatgcctttgta
tcatgctattgcttcccgtatggctttcattttctcctccttgtataaatcctggttgctgt
ctctttatgaggagttgtggcccgttgtcaggcaacgtggcgtggtgtgcactgtgtttgct
gacgcaacccccactggttggggcattgccaccacctgtcagctcctttccgggactttcgc
tttccccctccctattgccacggcggaactcatcgccgcctgccttgcccgctgctggacag
gggctcggctgttgggcactgacaattccgtggtgttgtcggggaaatcatcgtcctttcct
tggctgctcgcctgtgttgccacctggattctgcgcgggacgtccttctgctacgtcccttc
ggccctcaatccagcggaccttccttcccgcggcctgctgccggctctgcggcctcttccgc
gtcttcgccttcgccctcagacgagtcggatctccctttgggccgcctccccgcgtcgactt
taagaccaatgacttacaaggcagctgtagatcttagccactttttaaaagaaaagggggga
ctggaagggctaattcactcccaacgaagacaagatctgctttttgcttgtactgggtctct
ctggttagaccagatctgagcctgggagctctctggctaactagggaacccactgcttaagc
ctcaataaagcttgccttgagtgcttcaagtagtgtgtgcccgtctgttgtgtgactctggt
aactagagatccctcagacccttttagtcagtgtggaaaatctctagcagtacgtatagtag
ttcatgtcatcttattattcagtatttataacttgcaaagaaatgaatatcagagagtgaga
ggaacttgtttattgcagcttataatggttacaaataaagcaatagcatcacaaatttcaca
aataaagcatttttttcactgcattctagttgtggtttgtccaaactcatcaatgtatctta
tcatgtctggctctagctatcccgcccctaactccgcccatcccgcccctaactccgcccag
ttccgcccattctccgccccatggctgactaattttttttatttatgcagaggccgaggccg
cctcggcctctgagctattccagaagtagtgaggaggcttttttggaggcctagggacgtac
ccaattcgccctatagtgagtcgtattacgcgcgctcactggccgtcgttttacaacgtcgt
gactgggaaaaccctggcgttacccaacttaatcgccttgcagcacatccccctttcgccag
ctggcgtaatagcgaagaggcccgcaccgatcgcccttcccaacagttgcgcagcctgaatg
gcgaatgggacgcgccctgtagcggcgcattaagcgcggcgggtgtggtggttacgcgcagc
gtgaccgctacacttgccagcgccctagcgcccgctcctttcgctttcttcccttcctttct
cgccacgttcgccggctttccccgtcaagctctaaatcgggggctccctttagggttccgat
ttagtgctttacggcacctcgaccccaaaaaacttgattagggtgatggttcacgtagtggg
ccatcgccctgatagacggtttttcgccctttgacgttggagtccacgttctttaatagtgg
actcttgttccaaactggaacaacactcaaccctatctcggtctattcttttgatttataag
ggattttgccgatttcggcctattggttaaaaaatgagctgatttaacaaaaatttaacgcg
aattttaacaaaatattaacgcttacaatttaggtggcacttttcggggaaatgtgcgcgga
acccctatttgtttatttttctaaatacattcaaatatgtatccgctcatgagacaataacc
ctgataaatgcttcaataatattgaaaaaggaagagtatgagtattcaacatttccgtgtcg
cccttattcccttttttgcggcattttgccttcctgtttttgctcacccagaaacgctggtg
aaagtaaaagatgctgaagatcagttgggtgcacgagtgggttacatcgaactggatctcaa
cagcggtaagatccttgagagttttcgccccgaagaacgttttccaatgatgagcactttta
aagttctgctatgtggcgcggtattatcccgtattgacgccgggcaagagcaactcggtcgc
cgcatacactattctcagaatgacttggttgagtactcaccagtcacagaaaagcatcttac
ggatggcatgacagtaagagaattatgcagtgctgccataaccatgagtgataacactgcgg
ccaacttacttctgacaacgatcggaggaccgaaggagctaaccgcttttttgcacaacatg
ggggatcatgtaactcgccttgatcgttgggaaccggagctgaatgaagccataccaaacga
cgagcgtgacaccacgatgcctgtagcaatggcaacaacgttgcgcaaactattaactggcg
aactacttactctagcttcccggcaacaattaatagactggatggaggcggataaagttgca
ggaccacttctgcgctcggcccttccggctggctggtttattgctgataaatctggagccgg
tgagcgtgggtctcgcggtatcattgcagcactggggccagatggtaagccctcccgtatcg
tagttatctacacgacggggagtcaggcaactatggatgaacgaaatagacagatcgctgag
ataggtgcctcactgattaagcattggtaactgtcagaccaagtttactcatatatacttta
gattgatttaaaacttcatttttaatttaaaaggatctaggtgaagatcctttttgataatc
tcatgaccaaaatcccttaacgtgagttttcgttccactgagcgtcagaccccgtagaaaag
atcaaaggatcttcttgagatcctttttttctgcgcgtaatctgctgcttgcaaacaaaaaa
accaccgctaccagcggtggtttgtttgccggatcaagagctaccaactctttttccgaagg
taactggcttcagcagagcgcagataccaaatactgttcttctagtgtagccgtagttaggc
caccacttcaagaactctgtagcaccgcctacatacctcgctctgctaatcctgttaccagt
ggctgctgccagtggcgataagtcgtgtcttaccgggttggactcaagacgatagttaccgg
ataaggcgcagcggtcgggctgaacggggggttcgtgcacacagcccagcttggagcgaacg
acctacaccgaactgagatacctacagcgtgagctatgagaaagcgccacgcttcccgaagg
gagaaaggcggacaggtatccggtaagcggcagggtcggaacaggagagcgcacgagggagc
ttccagggggaaacgcctggtatctttatagtcctgtcgggtttcgccacctctgacttgag
cgtcgatttttgtgatgctcgtcaggggggcggagcctatggaaaaacgccagcaacgcggc
ctttttacggttcctggccttttgctggccttttgctcacatgttctttcctgcgttatccc
ctgattctgtggataaccgtattaccgcctttgagtgagctgataccgctcgccgcagccga
acgaccgagcgcagcgagtcagtgagcgaggaagcggaagagcgcccaatacgcaaaccgcc
tctccccgcgcgttggccgattcattaatgcagctggcacgacaggtttcccgactggaaag
cgggcagtgagcgcaacgcaattaatgtgagttagctcactcattaggcaccccaggcttta
cactttatgcttccggctcgtatgttgtgtggaattgtgagcggataacaatttcacacagg
aaacagctatgaccatgattacgccaagcgcgcaattaaccctcactaaagggaacaaaagc
tggagctgcaagc

What is claimed is:

1. A nucleic acid comprising:
(i) a first promoter operably linked to a retron nucleic acid sequence comprising:
an msr sequence;
an msd sequence;
a donor sequence within the msd sequence; and
a first inverted repeat sequence and a second inverted repeat sequence; and
(ii) a second promoter operably linked to a nucleic acid sequence encoding a guide RNA region.

2. The nucleic acid of claim 1, wherein the first promoter comprises an RNA polymerase II (Pol II) promoter, and the second promoter comprises an RNA polymerase III (Pol III) promoter.

3. The nucleic acid of claim 1 or 2, further comprising a stabilizing 5′ ribozyme sequence located 3′ of the first promoter and 5′ of the first inverted repeat sequence; a stabilizing 3′ ribozyme sequence located 3′ of the second inverted repeat sequence; and/or a long non-coding RNA (lncRNA) transcript located 3′ of the second inverted repeat sequence.

4. The nucleic acid of claim 3, wherein the stabilizing 5′ or 3′ ribozyme sequence is selected from the group consisting of a hammerhead ribozyme (HHR), a hepatitis delta virus (HDV) ribozyme, and a RiboJ ribozyme sequence.

5. The nucleic acid of claim 3, wherein the stabilizing 3′ sequence comprises the 3′ triple-helix and tRNA-like processing components of the lncRNA transcript Metastasis Associated Lung Adenocarcinoma Transcript 1 (MALATI).

6. The nucleic acid of claim 1, further comprising a transcription terminator sequence located 3′ of the second inverted repeat sequence and 5′ of the second promoter.

7. The nucleic acid of claim 1, wherein the donor sequence comprises a template for homology-directed repair (HDR).

8. A nucleic acid comprising a transcription unit comprising
a promoter operably linked to a retron nucleic acid sequence encoding a retron RNA and a nucleic acid sequence encoding a guide RNA region, wherein the retron nucleic acid sequence comprises:
an msr sequence;
an msd sequence;
a donor sequence within the msd sequence; and
a first inverted repeat sequence and a second inverted repeat sequence.

9. The nucleic acid of claim 8, wherein expression of the nucleic acid sequence results in a transcript comprising the retron RNA and guide RNA region, wherein the retron RNA and guide RNA region are separated after transcription by an RNA processing enzyme.

10. The nucleic acid of claim 9, wherein the RNA processing enzyme is a ribozyme or an endoribonuclease.

11. The nucleic acid of claim 8, wherein the nucleic acid sequence encoding the retron RNA is located S′ of the nucleic acid sequence encoding the guide RNA region.

12. The nucleic acid of claim 8, wherein the promoter comprises an RNA polymerase II (Pol II) promoter or an RNA polymerase III (Pol III) promoter.

13. An expression plasmid comprising the nucleic acid of claim 1.

14. A system for introducing a genetic modification at a target DNA locus, comprising:
i) the expression plasmid of claim 13;
ii) a second expression plasmid comprising a nucleic acid sequence encoding a CRISPR-associated endonuclease; and
iii) a third expression plasmid comprising a nucleic acid sequence encoding a reverse transcriptase.

15. The system of claim 14, wherein the second and third expression plasmids comprise a promoter operably linked to the nucleic acid sequences encoding the CRISPR-associated endonuclease and the reverse transcriptase, respectively.

16. The system of claim 15, wherein the promoter is a Pol II promoter.

17. The system of claim 14, wherein the CRISPR-associated endonuclease generates a single-stranded nick in one strand of the target DNA locus instead of a double-strand break (DSB).

18. The system of claim 14, wherein CRISPR-associated endonuclease comprises a nickase selected from the group consisting of nCas9-D10A and nCas9-H840A.

19. The system of claim 14, wherein the reverse transcriptase is selected from the group consisting of RT-Ec73, RT-St85, RT-Ec83, RT-Ec78, RT-Eco9, RT-Eco10, and RT-Eco11.

20. The system of claim 14, wherein the reverse transcriptase is RT-Ec73.

21. A method for editing DNA at a target locus in a cell, comprising introducing the system of claim 14 into the cell.

22. The method of claim 21, wherein the editing efficiency at the target locus is increased compared to a system comprising an expression plasmid wherein transcription products of the retron and the gRNA coding region are physically coupled.

23. The method of claim 21 or 22, wherein the cell is a eukaryotic cell.

24. The method of claim 23, wherein the cell is selected from the group consisting of a yeast cell, plant cell, mammalian cell, mammalian cell line, human cell, and human cell line.

25. A method of treating a genetic disease in a subject in need thereof, the method comprising administering to the subject an effective amount of
a) the nucleic acid of claim 1, the plasmid of claim 13, the system of claim 14, or a combination thereof;
b) a reverse transcriptase or a nucleic acid encoding the same, and
c) a sequence-specific endonuclease or a nucleic acid encoding the same.

26. The method of claim 25, wherein the reverse transcriptase is selected from the group consisting of RT-Ec73, RT-St85, RT-Ec83, RT-Ec78, RT-Eco9, RT-Eco10, and RT-Eco11.

27. The method of claim 25 or 26, wherein the reverse transcriptase is RT-Ec73.

28. The method of claim 25, wherein the sequence-specific endonuclease does not catalyze a double strand break in the DNA of a host cell in the subject.

29. The method of claim 25, wherein the sequence-specific endonuclease generates a single-stranded nick in one strand of a target DNA locus in a host cell of the subject.

30. The method of claim 25, wherein the sequence-specific endonuclease is a nickase.

31. The method of claim 30, wherein the nickase is nCas9-D10A or nCas9-H840A.

32. The method of claim 31, wherein the nickase is nCas9-H840A.

33. A pharmaceutical composition comprising:
(a) the nucleic acid of claim 1, the plasmid of claim 13, or the system of claim 14, or a combination thereof, and
(b) a pharmaceutically acceptable carrier.

34. A method for preventing or treating a genetic disease in a subject, the method comprising administering to the subject an effective amount of the pharmaceutical composition of claim 33 to correct a mutation in a target gene associated with the genetic disease.

35. The method of claim 25 or 34, wherein the genetic disease is selected from the group consisting of X-linked severe combined immune deficiency, sickle cell anemia, thalassemia, hemophilia, neoplasia, cancer, age-related macular degeneration, schizophrenia, trinucleotide repeat disorders, fragile X syndrome, prion-related disorders, amyotrophic lateral sclerosis, drug addiction, autism, Alzheimer's disease, Parkinson's disease, cystic fibrosis, blood and coagulation diseases and disorders, inflammation, immune-related diseases and disorders, metabolic diseases and disorders, liver diseases and disorders, kidney diseases and disorders, muscular/skeletal diseases and disorders, neurological and neuronal diseases and disorders, cardiovascular diseases and disorders, pulmonary diseases and disorders, ocular diseases and disorders, and a combination thereof.

36. A kit for modifying one or more target nucleic acids of interest at one or more target loci within a genome of a host cell, the kit comprising the nucleic acid of claim 1, or the plasmid of claim 13.

37. The kit of claim 36, further comprising a reverse transcriptase or a nucleic acid encoding the same, and a sequence-specific endonuclease or a nucleic acid encoding the same.

38. The kit of claim 37, wherein the reverse transcriptase is selected from the group consisting of RT-Ec73, RT-St85, RT-Ec83, RT-Ec78, RT-Eco9, RT-Eco10, and RT-Eco11.

39. The kit of claim 37 or 38, wherein the sequence-specific endonuclease comprises a nickase selected from the group consisting of nCas9-D10A and nCas9-H840A.

40. The kit of claim 36, further comprising a host cell.

41. The kit of claim 40, wherein the host cell is a eukaryotic cell.

42. The kit of claim 41, wherein the host cell is selected from the group consisting of a yeast cell, a plant cell, mammalian cell, mammalian cell line, human cell, and human cell line.

43. The kit of claim 36, further comprising one or more reagents for introducing the nucleic acids, plasmids, reverse transcriptase or sequence-specific endonuclease into the host cell.

44. A method for producing a retron RNA and a guide RNA, the method comprising:
i) contacting the nucleic acid of claim 8 with an RNA polymerase to produce a single transcript comprising the retron RNA and guide RNA sequences, and
ii) contacting the single transcript with an RNA processing enzyme that cleaves the transcript between the retron RNA and guide RNA sequences,
thereby producing the retron RNA and the guide RNA.

45. The method of claim 44, wherein the RNA processing enzyme is a ribozyme or an endoribonuclease.

46. The method of claim 44 or 45, wherein the nucleic acid sequence encoding the retron RNA is located 5′ of the nucleic acid sequence encoding the guide RNA.
